SEQUENCE LISTING (16 sequences)

SEQ ID NO: 1 - protein, 20 aa, Synthetic construct
GGTYSCHFGPLTWVCKPQGG

SEQ ID NO: 2 - protein, 5 aa, Synthetic construct
REGRT

SEQ ID NO: 3 - protein, 7 aa, Synthetic construct
SVVYGLR

SEQ ID NO: 4 - protein, 7 aa, Synthetic construct
QHREDGS

SEQ ID NO: 5 - protein, 4 aa, Synthetic construct
RGDS

SEQ ID NO: 6 - protein, 12 aa, Synthetic construct
GLLSGINAEWPC

SEQ ID NO: 7 - protein, 9 aa, Synthetic construct
VVVAAAEEE

SEQ ID NO: 8 - protein, 29 aa, Synthetic construct
  REPEAT 15..29: note = c is a value of
VVVAAAEEEGGTYSCHFGPLTWVCKPQGG

SEQ ID NO: 9 - protein, 18 aa, Synthetic construct
VVVAAAEEEGGGGREGRT

SEQ ID NO: 10 - protein, 4 aa, Synthetic construct
GGGG

SEQ ID NO: 11 - protein, 18 aa, Synthetic construct
VVVAAAEEEGGSVVYGLR

SEQ ID NO: 12 - protein, 18 aa, Synthetic construct
VVVAAAEEEGGQHREDGS

SEQ ID NO: 13 - protein, 17 aa, Synthetic construct
VVVAAAEEEGGGGRGDS

SEQ ID NO: 14 - protein, 23 aa, Synthetic construct
VVVAAAEEEGGGLLSGINAEWPC

SEQ ID NO: 15 - protein, 10 aa, Synthetic construct
VVVAAAEEEK

SEQ ID NO: 16 - protein, 12 aa, Synthetic construct
VVVAAAEEEECG

(19) United States
(12) Patent Application Publication
(10) Pub. No.: US 2025/0249144 A1
(43) Pub. Date: Aug. 7, 2025

(54) PEPTIDE AMPHIPHILE SUPRAMOLECULAR POLYMERS FOR SPRAY DELIVERY AND TISSUE REGENERATION APPLICATIONS

(71) Applicants: Tristan Clemons, Hattiesburg, MS (US); Penelope Jankoski, Hattiesburg, MS (US); Loria DiMartino, Covington, LA (US)

(72) Inventors: Tristan Clemons, Hattiesburg, MS (US); Penelope Jankoski, Hattiesburg, MS (US); Loria DiMartino, Covington, LA (US)

(73) Assignee: The University of Southern Mississippi, Hattiesburg, MS (US)

(21) Appl. No.: 19/047,480

(22) Filed: Feb. 6, 2025

Related U.S. Application Data
(60) Provisional application No. 63/550,726, filed on Feb. 7, 2024.

(51) Int. Cl. A61L 27/22; A61L 27/38; A61L 27/54; C08L 89/00

(52) U.S. Cl. CPC A61L 27/22; A61L 27/38; A61L 27/54; C08L 89/00; A61L 2300/252; A61L 2400/12; A61L 2430/34

(57) ABSTRACT

A composition including a plurality of non-covalently bonded peptide amphiphiles. The peptide amphiphiles include a hydrophobic alkyl tail moiety, a hydrophobic peptide portion including one or more hydrophobic amino acid residues, and a hydrophilic portion including one or more hydrophilic amino acid residues. Some or all of the peptide amphiphiles may optionally be functionalized with one or more bioactive components. Also disclosed is a A supramolecular polymer nanofiber scaffold comprising a plurality of the non-covalently bonded peptide amphiphiles, a kit including the peptide amphiphiles and a delivery device, and a method for promoting skin cell tissue regeneration by preparing peptide amphiphiles, optionally, collecting a biological material, mixing an aqueous composition, and the optional biological material with the peptide amphiphiles to produce an aqueous suspension or solution, and administering the suspension or a solution to a patient using high shear conditions.

### CROSS REFERENCE TO RELATED APPLICATIONS

This application claims the benefit of U.S. provisional application No. 63/550,726, filed on Feb. 7, 2024, the disclosure of which is hereby incorporated by reference in its entirety as if fully set forth herein.

### INCORPORATION OF MATERIAL OF XML SEQUENCE LISTING BY REFERENCE

The sequence listing submitted herewith as an XML file named “USM1023USSequenceListing” created on Feb. 6, 2025, which is 16 kilobytes in size, is hereby incorporated by reference in its entirety.

### BACKGROUND

Skin may be described as a bilayer structure with the outer barrier layer, the epidermis, being a constantly renewing layer. The dermis is a thicker, mechanically strong layer composed of an upper portion (papillary) and a lower portion (reticular). Loss of the epidermis and even superficial dermis can often heal with minimal scarring. An experimental incisional scar model in humans showed that incisions made at a depth of 0.53 mm or less (approximately the top ⅓ of the dermis) showed no long-term visible scar.¹

In contrast, dermal injury is associated with wound contraction and scarring. Deep partial thickness, and full-thickness traumatic wounds, such as those sustained from serious burns, heal primarily with scarring and wound contraction. It is these wounds that provide the biggest challenge to the clinician.

One example that supports clinicians in healing burn wounds is the ReCell® platform, an FDA approved device for the treatment of certain burn injuries that enables surgeons to produce a suspension of skin cells using a small sample of the patient's own skin and then spray the suspension over a wound bed for skin regeneration (prior art FIGS. 1A and 1B). This suspension contains the cells necessary to regenerate natural, healthy skin and is prepared and applied at the point of care in as little as 30-minutes.² The technology can take a skin sample equivalent to the size of a postage stamp and effectively treat a burn injury having a surface area equivalent to an A4 piece of paper to promote skin regeneration. Reports of positive outcomes using ReCell® appear in the burns and chronic wounds literature.³⁻⁶ This technology was first tested at the Bali bombings of 2002 in a mass casualty incident, and it was later lauded for being integral for patient survival, reduced incidence of scarring, and enhanced functionality.⁷ Despite this success, challenges remain with use of the ReCell® technology for ensuring that the sprayed cells remain localized at the wound.

According to a recent U.S. Department of Defense International State-of-the-Science Meeting on mitigating the effects of blast-related burn injuries, approximately 5 to 20 percent of combat-related casualties during Operation Iraqi Freedom and Operation Enduring Freedom included severe burns.⁸ Observational research conducted between 2009 and 2011 in Afghanistan suggests that improvised explosive devices (IEDs) accounted for as much as 87 percent of all burns.⁹ IED-related burns have an elevated risk of infection because they are usually contaminated with dirt and debris, and are often associated with multiple casualties.¹⁰ However, immediate evacuation might not always be possible. In these cases, burn injuries must be managed in a prolonged field care environment, by non-experts and beyond doctrinal planning timelines, until the patient can be delivered to definitive care, presenting challenges unique to military populations.

Thus, there exists an urgent need for an intervention amenable to rapid field deployment and application by non-experts, that provides enhanced skin regeneration, and faster wound closure following burn injuries and which is applicable to both civilian and military populations.

Current treatment options for healing skin wounds are suboptimal—despite the significant social and financial burden wound healing imposes on society. Numerous regenerative scaffolds and skin substitutes have been proposed for the management of skin injuries following burns, including both biological (autologous, allogeneic and xenogeneic), and synthetic (biodegradable and non-biodegradable) regeneration scaffolds.” Despite the wide range of skin tissue regeneration products that are commercially available, each is lacking in certain aspects. Some of the major deficiencies of current therapeutic interventions include fragility of the scaffold, risk of infection, risk of graft failure, prolonged processing times, and high costs.^{3, 12} Similar to biomaterial interventions, growth factors, cytokines, and chemokines have also been extensively explored for tissue regeneration applications, especially for coordinating multiple cell interactions responsible for achieving wound healing. However, especially topical growth factor delivery to skin wounds must be resistant to rapid degradation from the wounds' proteolytic environment and include means of localization at the site of injury to avoid off target side effects.¹³ Peptide based therapeutics that can mimic the effects of these important biomolecules, while resisting proteolytic degradation, can potentially overcome these drawbacks associated with protein therapies. Further, locating these bioactive materials onto a scaffold that mimics both growth factor activity and the architecture of lost extracellular matrix provides the added advantage of limiting these powerful stimulating effects to the site where regeneration is required.

Finally, biomaterial interventions for skin regeneration need to be effective, rapidly delivered, and relatively low cost. The ReCell® platform has demonstrated the capability of achieving this through spraying a patient's own skin cells over a wound to encourage wound healing following acute burns (prior art FIG. 1). This manual spray delivery provides an intervention that is fast and easy to administer, an important consideration in avoiding infection. It is also inexpensive and does not require follow up surgery for scaffold removal. Despite these advantages the opportunity exists to support cells with a synthetic scaffold that can be sprayed with the cells, improves cell localization, especially on non-uniform injury sites, and that provides appropriate regenerative cues to promote wound healing.

The current gold standard for full-thickness injuries is surgical debridement and closure with autologous split thickness skin grafts (STSGs).¹⁴ The donor site heals in a similar fashion to a superficial partial thickness wound, and once healed, can be used for further skin graft re-harvesting. Generally, the application of a thicker split thickness skin graft will result in less contraction of the wound because of the increased dermal support that it provides. However, procurement of such a thick skin grant causes a more significant injury at the donor site that will take longer to heal. Severe skin injuries, such as those from large burns, invariably lack sufficient skin donor sites for skin wound repair. As the percentage of body surface area affected increases, donor site availability decreases, and surgical treatment by this method becomes increasingly difficult. Autografts can be meshed, uniformly perforated, and stretched to cover larger areas of the wound, but the cosmetic and functional outcomes are inferior to standard STSGs. In addition, large donor tissue sites create additional wounds equivalent to second degree burns, further increasing the total body surface affected and the overall risk to the patient. These limitations have driven research toward alternative dermal and epidermal replacement technologies, using tissue engineering to create skin substitutes.

A number of scaffolds, and also cell:scaffold combinations currently exist for the treatment of skin injuries. These include Integra®, Alloderm® and Surederm® as dermal templates, as well as Dermagraft®, Apligraf® and Orcell® as combined cell:scaffold skin substitutes, among others.¹⁵ Current evidence supporting the efficacy of these treatments is limited, and many are restricted in use due to expense, low stability, poor take rates, and uncertainty over the use of allogeneic cells. For example, Integra® is a dermal regeneration template based on a thin silicon barrier bound to a complex matrix of animal derived collagen and proteoglycan matrix, to replicate lost dermal tissue. It has been extensively used for acute burn wounds, and more recently has been approved for diabetic foot ulcers.¹⁶ Despite the clear promise of Integra® in providing the mechanical scaffolding reminiscent of lost dermal tissue, it lacks the cell incorporation or bioactive stimuli to effectively promote regeneration beyond the innate remaining tissue. Further, a major drawback of Integra® is the requirement for follow up surgery to remove the silicone outer layer, resulting in added expense, time, and potential for infections to occur.¹⁵

Similarly, Dermagraft® is a cryopreserved human fibroblast derived dermal substitute, where dermal fibroblasts are cultured in vitro onto a bioabsorbable mesh secreting dermal collagen, matrix proteins, growth factors, and cytokines to create a scaffold containing both growth cues and metabolically active living cells.¹⁷ The incorporation of bioactivity within Dermagraft®, however, results in a high cost of the scaffold, a greater potential for wound infection, a relatively short shelf-life, and ultra-low (e.g. −80° C.) transport and storage temperature requirements.

Accordingly, an opportunity remains to improve wound healing by providing a sprayable and bioactive scaffold, engineered to support rapid skin regeneration and maintain cell localization, both important considerations in the treatment of burns, particularly in a mass casualty setting. The ideal next generation skin substitutes need to be biocompatible, biodegradable, easy to apply, economically viable, stable, transportable, have low or no immunogenicity, and must improve outcomes, both functional and aesthetic. Ease and speed of application are particularly important for debrided full-thickness injuries, to reduce blood loss and improve healing. Rapid treatment also has the significant advantage of providing surgical units with enhanced capability to deal with mass casualty situations where burn injuries are often encountered.

### SUMMARY OF THE INVENTION

The present invention provides a solution to these currently unmet needs discussed above by providing novel peptide amphiphiles including peptide amphiphiles functionalized with bioactive materials. These peptide amphiphiles can be sprayable to provide optionally bioactive, supramolecular polymers that form scaffolds. In some embodiments the peptide amphiphiles may be prepared and delivered in conjunction with a biological material.

The peptide amphiphiles (PAs) of the present invention are biocompatible, biodegradable, and possess the unique capability of self-healing following exposure to high shear, which provides a significantly improved material intervention when employed in a variety of applications that benefit from the provision of a scaffold, such as the application of cells for skin regeneration.

The present invention may be described by the following sentences:

1. In a first aspect, the present invention relates to a composition comprising:
a plurality of non-covalently bonded peptide amphiphiles, wherein each of the peptide amphiphiles comprises:
   a hydrophobic alkyl tail moiety,
   a hydrophobic peptide portion comprising one or more hydrophobic amino acid residues, and
   a hydrophilic portion comprising one or more hydrophilic amino acid residues, and
wherein about 0 mol % to about 100 mol % of the plurality of peptide amphiphiles are functionalized with one or more bioactive components and the one or more bioactive components are selected from the group consisting of bioactive epitope peptide sequence EMP1 (GGTYSCHFGPLTWVCKPQGG (SEQ ID NO.: 1)), bioactive epitope peptide ERK receptor activating REGRT (SEQ ID NO.2), bioactive epitope peptide sequence SVVYGLR (SEQ ID NO.: 3), bioactive epitope peptide sequence, QHREDGS (SEQ ID NO.4), cell adhesion peptide sequence, RGDS (SEQ ID NO: 5), bioactive wound healing peptide sequence, GLLSGINAEWPC (SEQ ID NO: 6), antioxidant moiety lipoic acid, antioxidant moiety protocatechuic acid (PCA), and antioxidant moiety glutathione tripeptide.

2. The composition of claim 1, wherein the peptide amphiphiles are in an aqueous solution.

3. The composition of sentence 1, may further comprise a biological material selected from the group consisting of biological fluids, biological cells, and biological tissues.

4. The composition of sentence 1, may further comprise a biological material selected from the group consisting of an autologous material, an allogeneic material, a xenogeneic material, or a biomimetic synthetic of biological material.

5. The composition of sentence 2, wherein the biological material may include autologous cells.

6. The composition of any one of sentences 1-5, may have a shear modulus of from about 2 Pa to about 20,000 Pa.

7. The composition of any one of sentences 1-6, wherein the hydrophobic alkyl tail moiety may be derived from one or more saturated or unsaturated fatty acids having from about 6 to 22 carbon atoms, or from about 8 to 20 carbon atoms.

8. The composition of any one of sentences 1-7, wherein the hydrophobic portion may include one or more hydrophobic amino acid residues of amino acids selected from the group consisting of glycine, alanine, valine, leucine, phenylalanine, cysteine, isoleucine, and combinations thereof.

9. The composition of any one of claims 1-8, wherein the hydrophilic portion may include one or more hydrophilic amino acid residues of amino acids selected from the group consisting of serine, asparagine, glutamine, threonine, arginine, lysine, histidine, aspartic acid, and glutamic acid and combinations thereof.

10. The composition of any one of claims 1-9, wherein the hydrophilic portion may further comprise a component selected from the group consisting of non-natural amino acids, and hydrophilic moieties without charge.

11. The composition of any one of sentences 1-10, wherein the hydrophobic alkyl tail moiety is a hexadecyl group, the one or more hydrophobic amino acid residues comprise alanine and valine, and the one or more hydrophilic amino acid residues comprise glutamic acid.

12. The composition of sentence 1, wherein one or more the peptide amphiphiles may be selected from the group consisting of

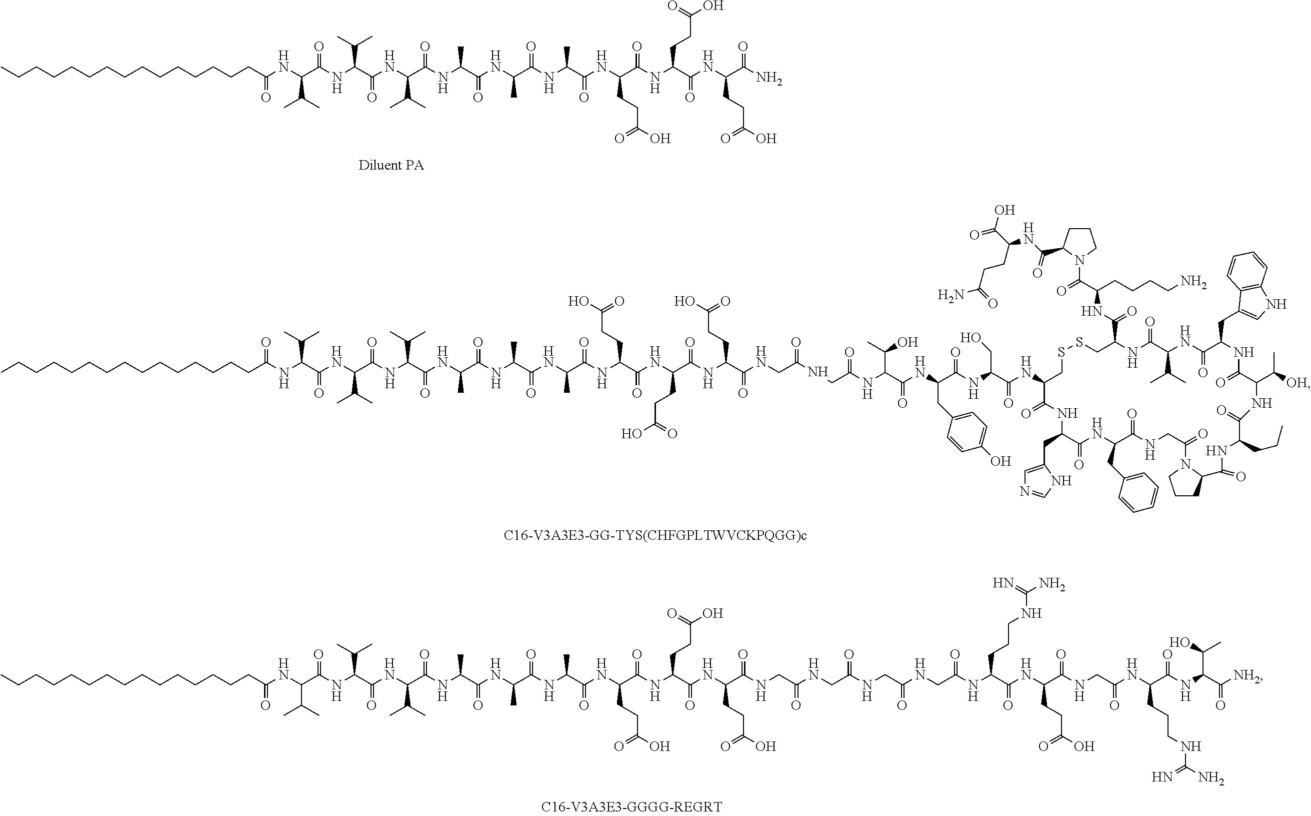

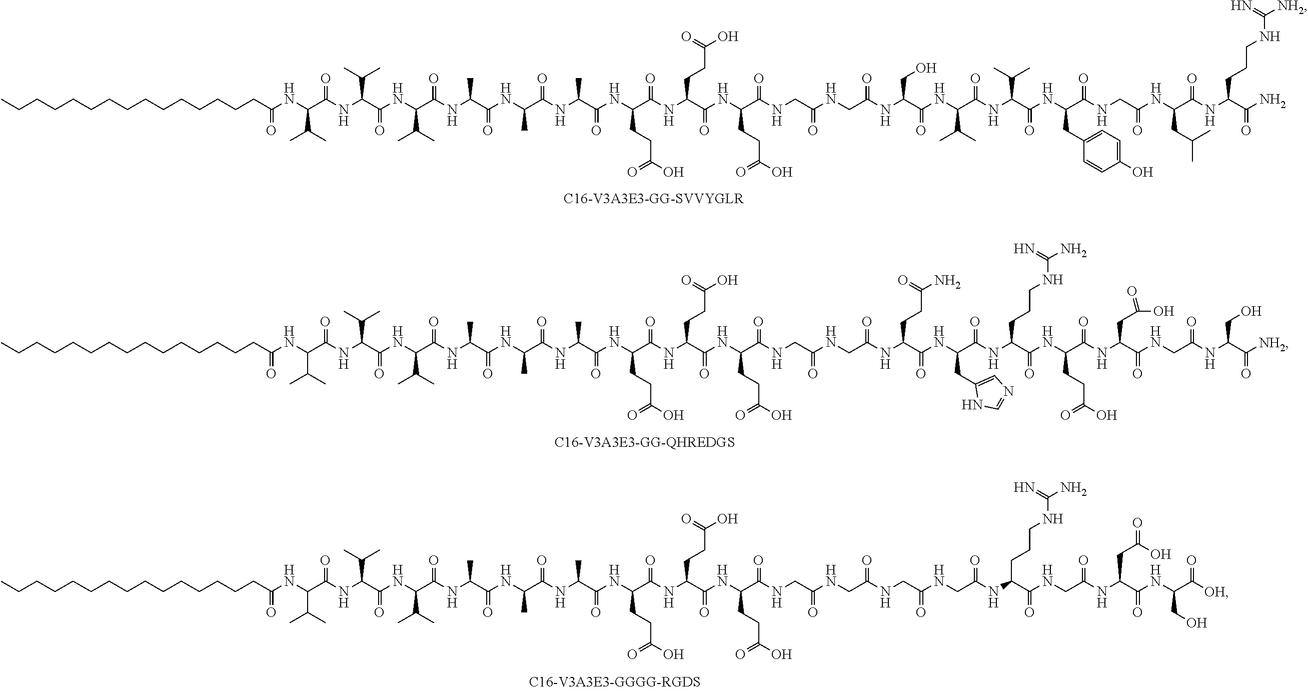

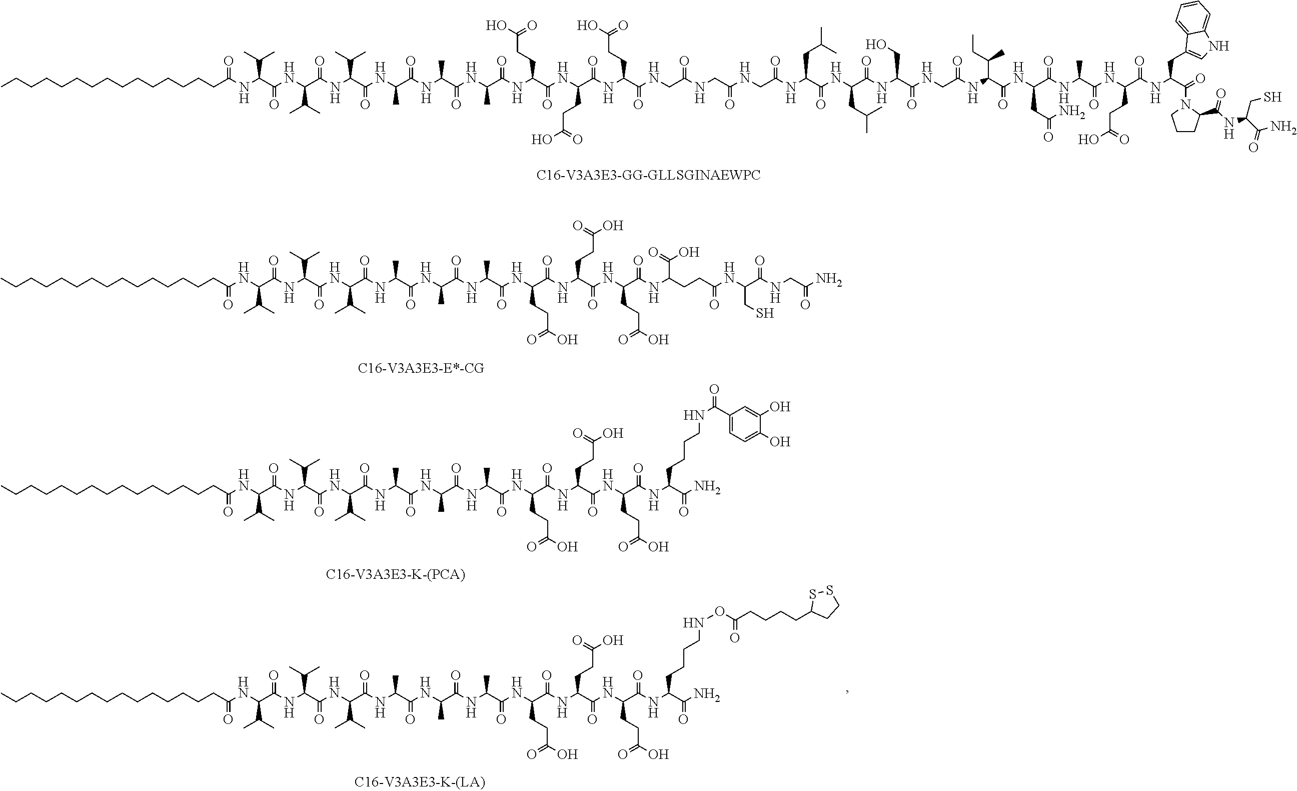

and combinations thereof.

13. The composition of any one of sentences 1-12, wherein about 5 mol % to about 100 mol %, or from about 5 mol % to about 80 mol %, or from about 5 mol % to about 70 mol %, or from about 10 mol % to about 50 mol %, or from about 10 mol % to about 40 mol %, or from about 10 mol % to about 100 mol %, or from about 20 mol % to about 100 mol %, or from about 40 mol % to about 100 mol %, or from about 60 mol % to about 100 mol %, or form about 80 mol % to about 100 mol %, or up to 90 mol %, of the plurality of peptide amphiphiles may be functionalized with the one or more bioactive components.

14. The composition of any one of sentences 1-13, wherein the composition is capable of forming a bioactive scaffold.

15. The composition of any one of sentences 1-14, wherein the composition comprises an aqueous buffer.

16. The composition of any one of sentences 1-15, wherein the composition is capable of forming a hydrogel in the presence of water.

17. The composition of any one of sentences 1-16, wherein the composition is a thixotropic fluid.

18. In a second aspect, the present invention relates to a supramolecular polymer nanofiber scaffold comprising a plurality of non-covalently bonded peptide amphiphiles as described in any one of sentences 1-17.

19. The supramolecular polymer nanofiber scaffold of sentence 18, wherein the peptide amphiphiles are selected from the group consisting:

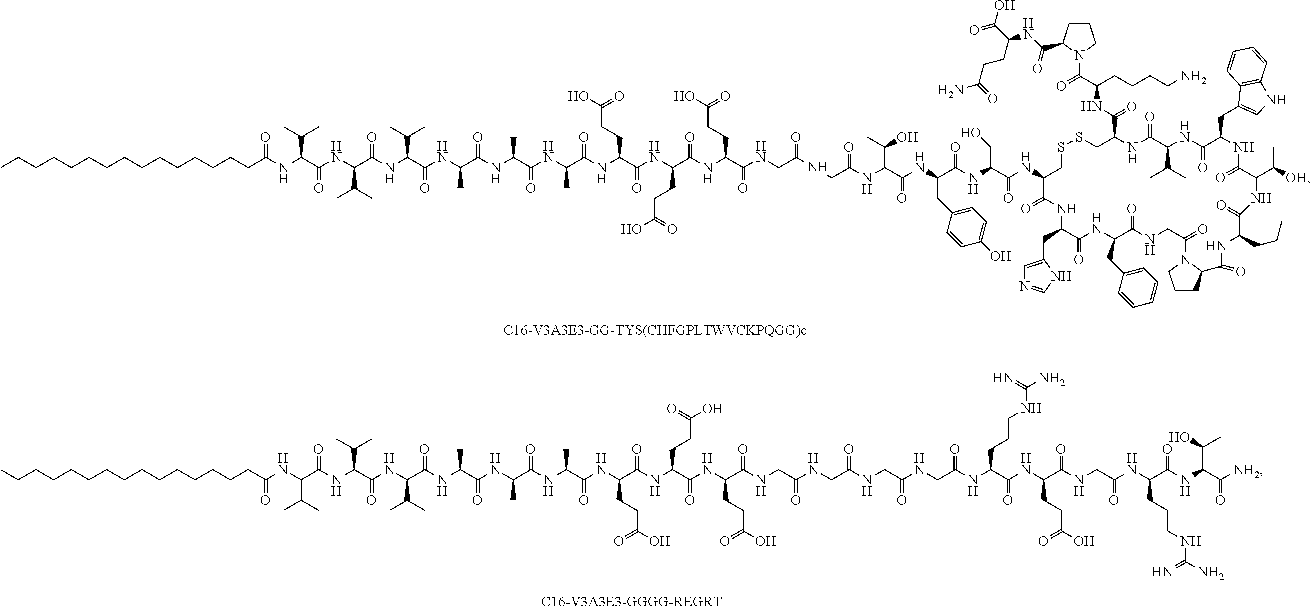

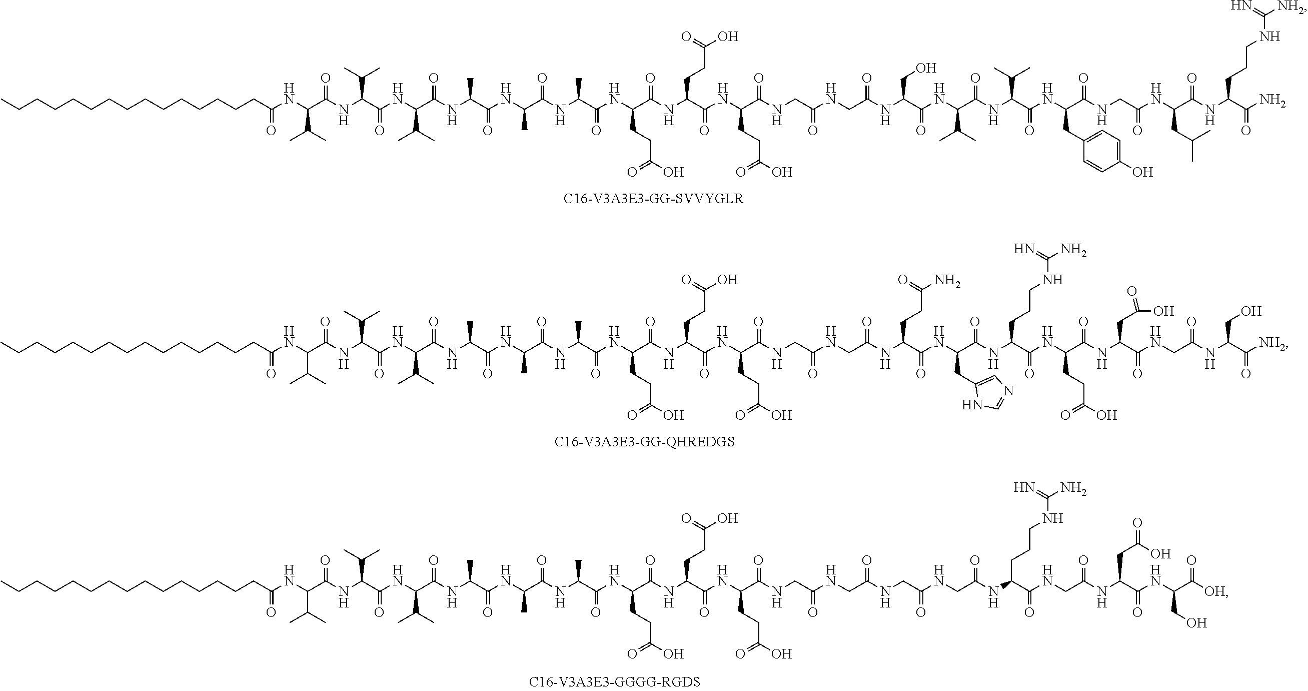

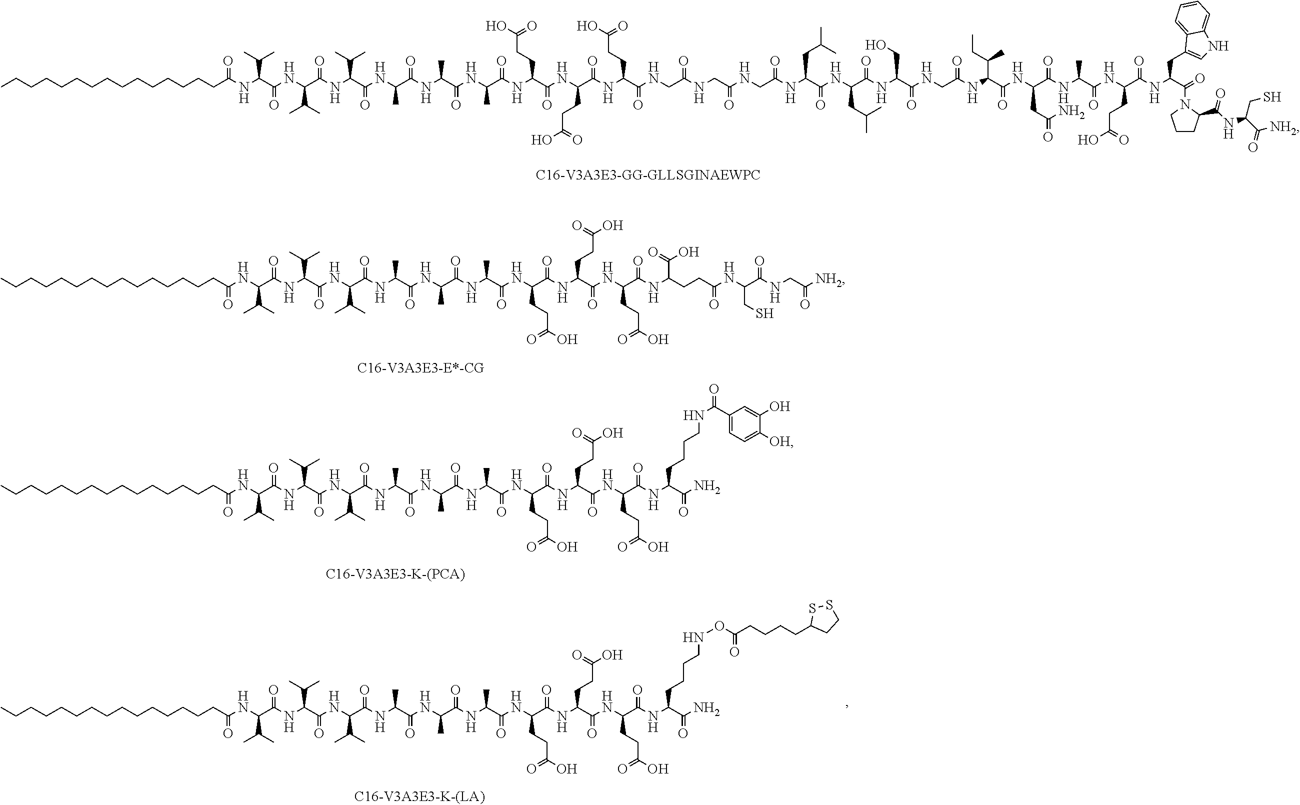

and combinations thereof.

20. The supramolecular polymer nanofiber scaffold of any one of sentences 18-19, wherein the supramolecular polymer nanofiber scaffold is configured to reversible disassemble in response to high shear conditions and to self-assemble when returned to lower shear conditions.

21. The composition of sentence 20, wherein the high shear conditions are from injection delivery, 3D printing, and spray delivery.

23. In a third aspect, the present invention relates to a kit comprising:
the composition of any one of sentences 1-17 and a delivery device selected from the group consisting of a syringe, dropper, a film, a 3D bioprinter, and a sprayer.

24. In a fourth aspect, the present invention relates to a method for promoting skin cell tissue regeneration comprising steps of:
preparing the composition of any one of sentences 1-17
optionally, collecting a biological material,
mixing an aqueous composition, and the optional biological material with the composition of any one of sentences 1-17 to produce an aqueous suspension or solution, and
administering the suspension or solution to a patient using high shear conditions.

### BRIEF DESCRIPTION OF THE DRAWINGS

FIG. 1A shows the prior art ReCell® kit which includes the components required for rapid donor skin collection and matrix digestion.

FIG. 1B shows a prior art spray delivery mechanism for the ReCell® kit for delivery of skin cells following a burn injury.

FIG. 2 shows the four major structural components used for forming the peptide amphiphile (PA) nanofiber of the present invention that is capable of both self-assembling and self-healing into a matrix. The alkyl tail (first segment, starting from the left) allows for hydrophobic collapse and aggregation of molecules in an aqueous environment. The R-sheet forming region (second component from the left), is a hydrophobic amino acid, for example, valine or alanine, which encourages strong beta-sheet hydrogen bonding among molecules. The hydrophilic portion (third component from the left) includes both hydrophilic amino acids, for example, glutamic acid, to provide amphiphilicity and allow for molecular solubility. The fourth component is optionally a bioactive epitope, for example, a bioactive peptide.

FIG. 3A shows the chemical structure of the bioactive and pro-regenerative peptide amphiphile (PA) molecules for use in spray delivery of bioactive supramolecular PA nanofibers.

FIG. 3B shows the co-assembly and supramolecular nanofiber formation.

FIG. 3C shows SEM images of PA nanofibers demonstrating that the PA nanofibers can self-heal to form a scaffold similar to a lost extracellular matrix following spray delivery.

FIG. 3D shows PA nanofiber cytotoxicity assessed using a lactate dehydrogenase assay and human embryonic kidney cells (HEK293) following 24 hours of incubation.

FIG. 3E shows cell viability 72 hrs. following ReCell® spray delivery with and without PA nanofibers.

FIG. 3F shows a photograph of viable HEK293 cells sprayed with the PA demonstrating improved adherence in the presence of the PA nanofibers.

FIG. 3G shows an assessment of the hydrogen bonding between molecules within the nanofibers for PA without a bioactive epitope (diluent PA), PA with REGRT, and PA with SVVYGLR (SV), using circular dichroism.

FIGS. 3H, 3I and 3J show the critical aggregation concentration, a measure of the propensity for the molecules to assemble together, using a Nile red spectrophotometric assay.

FIG. 3H shows the critical aggregation concentration for diluent PA, PA without a bioactive epitope.

FIG. 3I shows the critical aggregation concentration for PA functionalized with SVVYGLR (SEQ ID NO: 3) bioactive epitope.

FIG. 3J shows the critical aggregation concentration for PA functionalized with REGRT (SEQ ID NO: 2) bioactive epitope.

FIG. 4A shows a Cryogenic transmission electron microscopy micrograph of a peptide amphiphile at 1 min, following dissolution in aqueous media.

FIG. 4B shows a Cryogenic transmission electron microscopy micrograph of a peptide amphiphile at 5 min, following dissolution in aqueous media.

FIG. 4C shows a Cryogenic transmission electron microscopy micrograph of a peptide amphiphile at 10 min, following dissolution in aqueous media.

FIG. 4D shows a Cryogenic transmission electron microscopy micrograph of a peptide amphiphile at 30 min, following dissolution in aqueous media.

FIGS. 4A-4D demonstrate that the peptide amphiphiles are programmed for rapid self-assembly into nanofibers since self-assembled nanofibers are observed at 5 minutes following dissolution in aqueous media and after 10 minutes, these nanofibers have elongated to micron length scale.

FIG. 5 shows a schematic representation of an in vivo model of wound healing in a murine burn injury model.

FIG. 6 shows an example of data obtained from the in vivo murine burn injury model with the bioactive PA nanofibers.

FIG. 7 shows preliminary data from the pre-clinical porcine burn injury model with evidence of improved wound healing and scar outcomes in the presence of the bioactive PA nanofibers relative to controls.

FIG. 8 shows a schematic of the sprayable bioactive PA nanofibers as a room temperature stable, lyophilized, and sterile powder to be used with the ReCell® device, suitable for rapid buffer reconstitution and spray delivery with cells.

FIG. 9A shows viscosity studies to determine the concentration of PA that can be used without altering the spray pattern and deposition from the ReCell® nozzle. Comparative Spray Deposition Patterns as assessed with water sensitive Spot-on-Paper® are shown.

FIG. 9B shows cone-plate rheometer results comparing the viscosities of the FDA approved ReCell® solution and the suspension of the PA nanofibers of the present invention.

FIGS. 10A, 10B, and 10C show transmission electron microscopy (TEM) characterizations of bioactive PA nanofibers which confirm their self-assembly into fibrils.

FIG. 10A shows a cryogenic TEM image of the diluent PA.

FIG. 10B shows a conventional TEM image of the PA with the bioactive epitope REGRT (SEQ ID NO: 2).

FIG. 10C shows a conventional TEM image of the PA with the bioactive epitope SVVYGLR (SEQ ID NO: 3).

FIG. 11 shows the creation of a cell suspension with PA nanofibers capable of rapid spray delivery to an acute burn injury to enhance skin regeneration.

FIG. 12 is a plot showcasing the thixotropic properties of the composition of the present invention by stepwise straining the diluent PA under low strain of 0.01% and subsequent high strain of 400%. Modulus recovery following high strain shows material recovery and self-healing following large deformations.

FIGS. 13A-13H show toxicity and migration results after 24 hours of high concentration of diluent PA or 20% REGRT (RG) PA.

FIG. 13A shows cytotoxicity results for the control, diluent PA, and 20% REGRT PA (RG PA).

FIG. 13B shows a confocal microscopy z-stack of viable HEK293 cells within the functionalized PA nanofiber matrix after 24 hours for 4 mM of 20% RG PA.

FIG. 13C shows a confocal microscopy z-stack of viable HEK293 cells within the functionalized PA nanofiber matrix after 24 hours for 6 mM of 20% RG PA.

FIG. 13D shows a confocal microscopy z-stack of viable HEK293 cells within the functionalized PA nanofiber matrix after 24 hours for 10 mM of 20% RG PA.

FIG. 13E shows a confocal microscopy z-stack of viable HEK293 cells after 24 hours as a control (no PA matrix).

FIG. 13F shows a confocal microscopy z-stack of viable HEK293 cells within the PA nanofiber matrix after 24 hours for 4 mM of the diluent PA.

FIG. 13G shows a confocal microscopy z-stack of viable HEK293 cells within the PA nanofiber matrix after 24 hours for 6 mM of the diluent PA.

FIG. 13H shows a confocal microscopy z-stack of viable HEK293 cells within the PA nanofiber matrix after 24 hours for 10 mM of the diluent PA.

FIGS. 14A-14F show migration results for varying concentrations of diluent PA after 72 hours.

FIG. 14A shows a confocal microscopy z-stack of viable HEK293 cells within the PA nanofiber matrix for the control (no PA matrix).

FIG. 14B shows a confocal microscopy z-stack of viable HEK293 cells within the PA nanofiber matrix for 2 mM of diluent PA.

FIG. 14C shows a confocal microscopy z-stack of viable HEK293 cells within the PA nanofiber matrix for 4 mM of diluent PA.

FIG. 14D shows a confocal microscopy z-stack of viable HEK293 cells within the PA nanofiber matrix for 6 mM of diluent PA.

FIG. 14E shows a confocal microscopy z-stack of viable HEK293 cells within the PA nanofiber matrix for 8 mM of diluent PA.

FIG. 14F shows a confocal microscopy z-stack of viable HEK293 cells within the PA nanofiber matrix for 10 mM of diluent PA.

FIGS. 15A-15D show the spray viability of the samples after 48 hours.

FIG. 15A shows the spray viability of the control (no PA, No Spray) after 48 hours.

FIG. 15B shows the spray viability of the sprayed control (No PA) after 48 hours.

FIG. 15C shows the spray viability of a sample incubated with 5 mM Diluent PA after 48 hours.

FIG. 15D shows the spray viability of a sample sprayed with 5 mM Diluent PA after 48 hours.

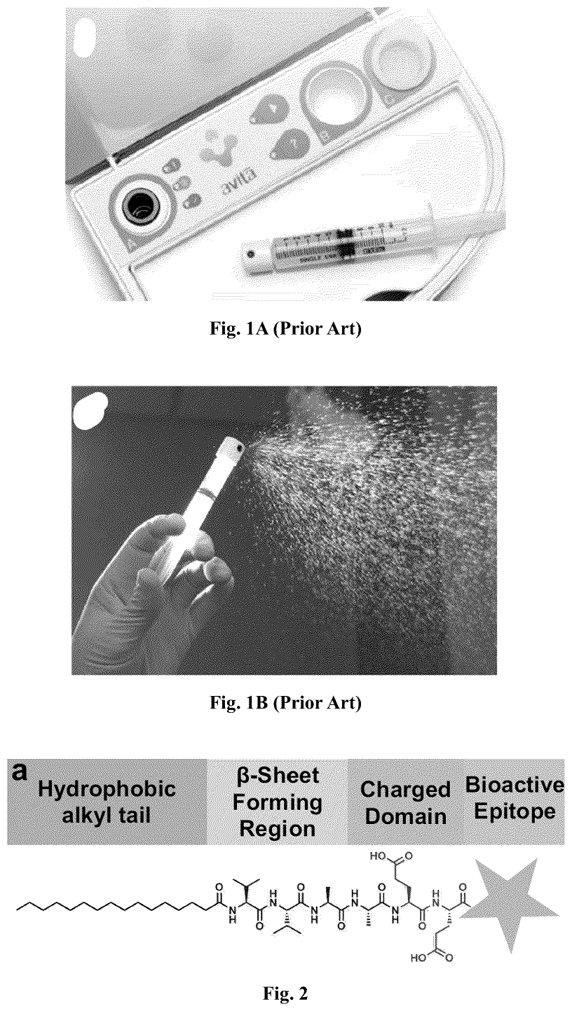

### DETAILED DESCRIPTION OF THE INVENTION

The present invention relates to a composition including:
a supramolecular polymer nanofiber comprising a plurality of non-covalently bonded peptide amphiphiles, wherein each of the peptide amphiphiles includes:
   a hydrophobic alkyl tail moiety,
   one or more hydrophobic amino acid residues, and
   one or more hydrophilic amino acid residues,
      wherein from about 0 mol % to about 100 mol % of the plurality of peptide amphiphiles are functionalized with one or more bioactive epitopes comprising a bioactive sequence selected from the group consisting of peptide sequence EMP1 (GGTYSCHFGPLTWVCKPQGG (SEQ ID NO.: 1), ERK receptor activating REGRT (SEQ ID NO.2), peptide sequence SVVYGLR (SEQ ID NO.: 3), peptide sequence QHREDGS (SEQ ID NO.4), cell adhesion peptide sequence, RGDS (SEQ ID NO: 5), and the bioactive wound healing peptide sequence, GLLSGINAEWPC (SEQ ID NO: 6).

The compositions of the present invention may include bioactive components, useful for forming bioactive scaffolds to promote, for example, skin tissue regeneration.

The composition of the present invention comprises a plurality of non-covalently bonded peptide amphiphiles. The composition forms a hydrogel scaffold. The scaffold composition may be formed in an aqueous environment to create the hydrogel. For example, the composition may be in the form of an aqueous solution or include an aqueous buffer and further includes one or more metal ions for forming the hydrogel scaffold. The presence of water and the metal ions facilitate nanofiber formation and ionic cross linking. The one or more metal ions may be selected from divalent metal ions, such as beryllium, magnesium, iron, cobalt, barium, strontium, calcium, zinc, and copper. Suitable aqueous buffers include sterile isotonic buffered solutions commonly used in medicine to maintain physiological conditions, support cellular function, and facilitate fluid replacement.

The compositions of the present invention are thixotropic, therefore, when the compositions are subject to high shear conditions, for example, when processed through a syringe, dropper, applied as a film or by a 3D bioprinter, or when sprayed through a spray nozzle, the hydrogel composition flows like a liquid as since the composition becomes less viscous. Once the composition is returned to a normal, lower shear condition, relative to the high shear condition, the composition self-assembles to reform the hydrogel scaffold. This is due to the supramolecular polymer nanofiber being configured to reversibly disassemble in response to high shear conditions and to self-assemble when returned to the lower shear condition. The reformation of the scaffold maintains the compositions of the present invention localized in a specific area of application.

### Supramolecular Polymer Nanofiber

The supramolecular polymer nanofiber of the present invention comprises a plurality of non-covalently bonded peptide amphiphiles, wherein each of the peptide amphiphiles comprises a hydrophobic alkyl tail moiety, a hydrophobic portion comprising one or more hydrophobic amino acid residues, and a hydrophilic portion comprising one or more hydrophilic amino acid residues. The peptide amphiphiles may also include a beta sheet forming region.

The supramolecular polymer nanofiber may be functionalized by functionalizing some or all of the peptide amphiphiles may be further functionalized with one or more bioactive components or bioactive epitopes. For example, about 0 mol % to about 100 mol %, or about 5 mol % to about 80 mol %, or from about 5 mol % to about 70 mol %, or from about 10 mol % to about 50 mol %, or from about 10 mol % to about 40 mol % or from about 10 mol % to about 100 mol %, or from about 20 mol % to about 100 mol %, or from about 40 mol % to about 100 mol %, or from about 60 mol % to about 100 mol %, or form about 80 mol % to about 100 mol %, or up to 90 mol %, of the total moles of the plurality of peptide amphiphiles may be functionalized with one or more bioactive components or bioactive epitopes. Therefore, approximately 0 mol % to about 95 mol %, or from about 20 mol % to about 95 mol %, or from about 30 mol % to about 95 mol %, or from about 50 mol % to about 90 mol % or from about 60 mol % to about 90 mol %, of the total moles of the peptide amphiphiles may optionally remain unfunctionalized.

Optionally, the supramolecular polymer nanofiber may comprise unfunctionalized fluorescently labeled peptide amphiphiles to provide the ability to fluorescently track the bioactive scaffold. The optional fluorescently labeled peptide amphiphiles, if present, may be present in an amount of from about 0.1 mol % to about 10 mol %, or from about 0.5 mol % to about 5 mol %, or from about 1 mol % to about 3 mol %, based on the total number of moles of the peptide amphiphiles.

### Peptide Amphiphiles

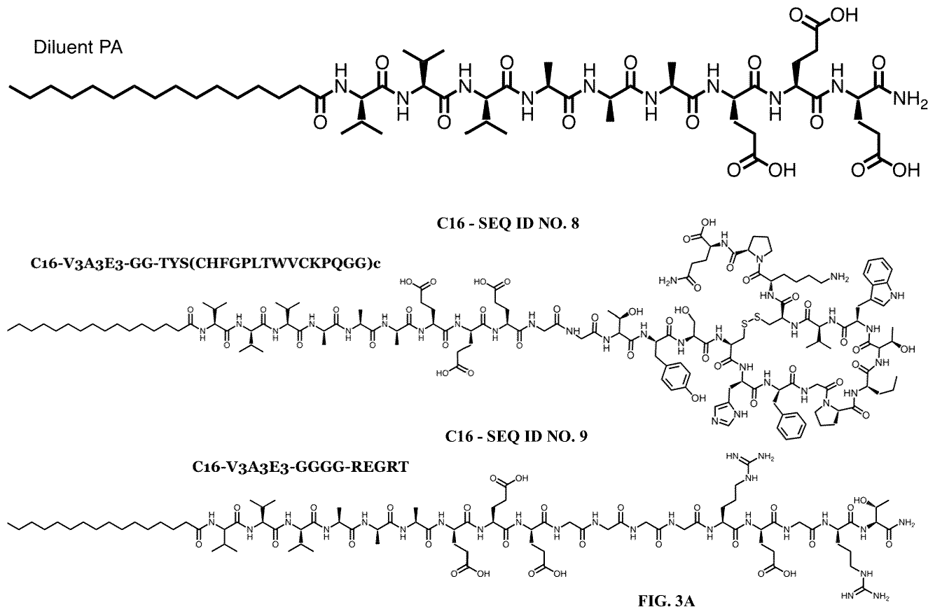

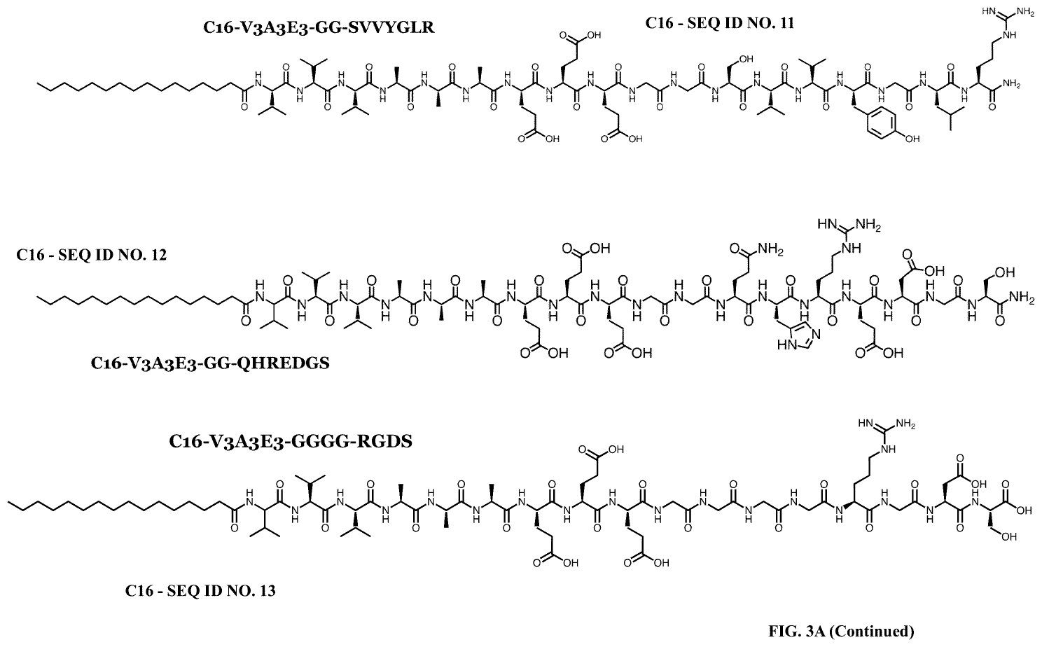

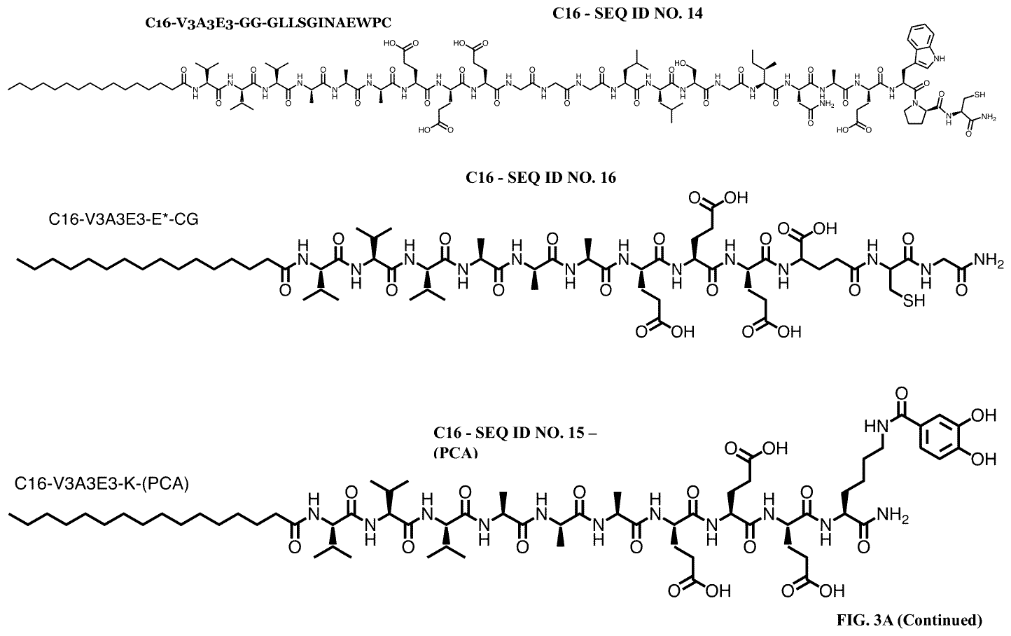

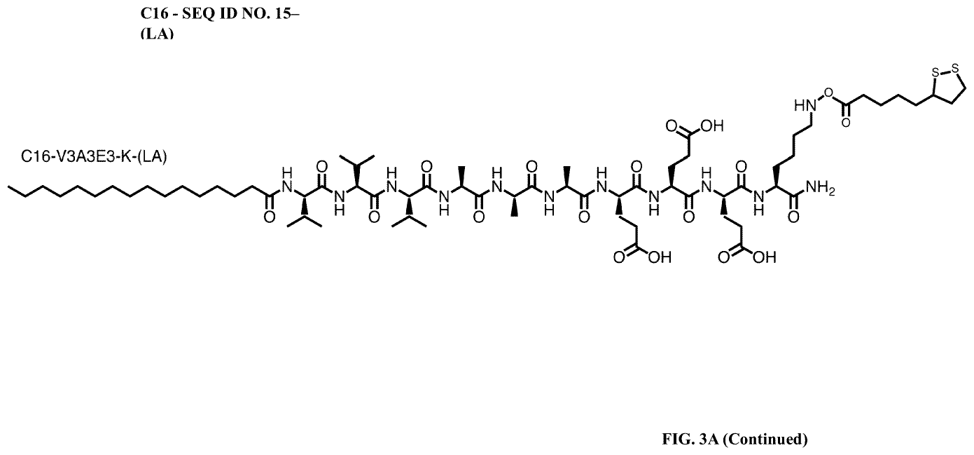

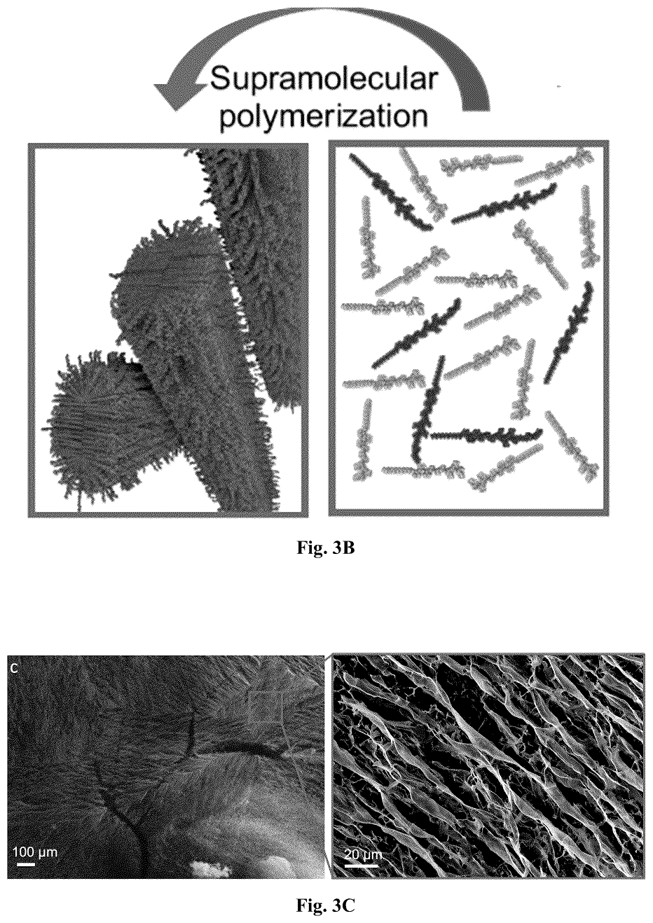

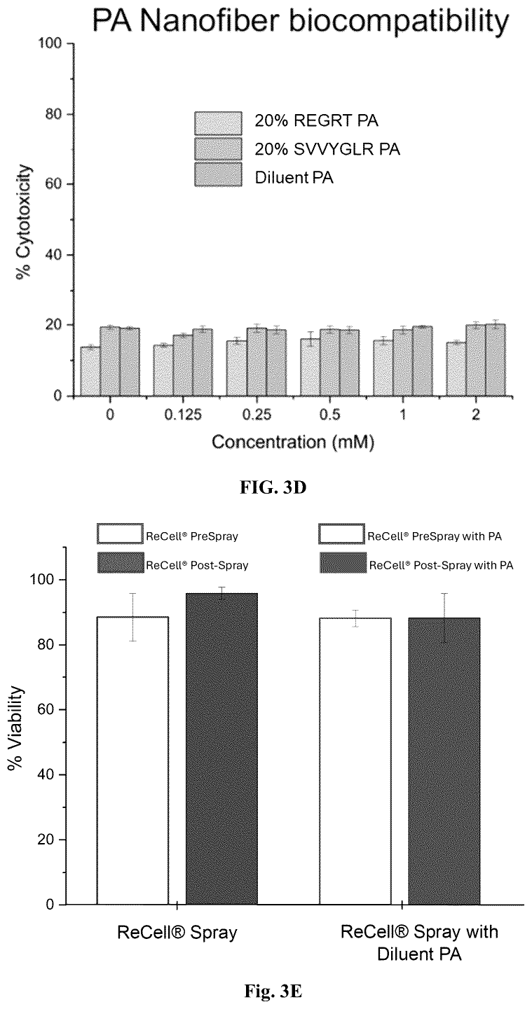

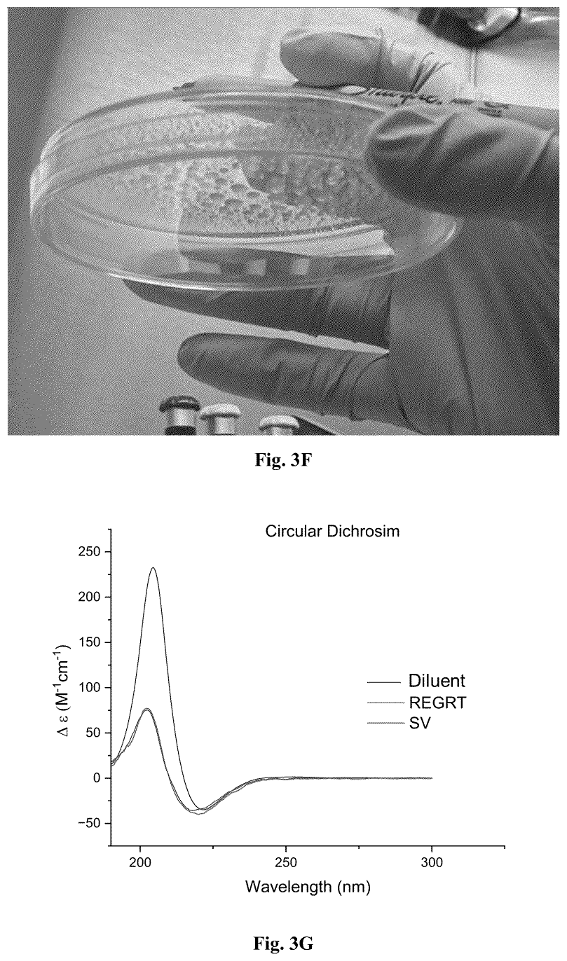

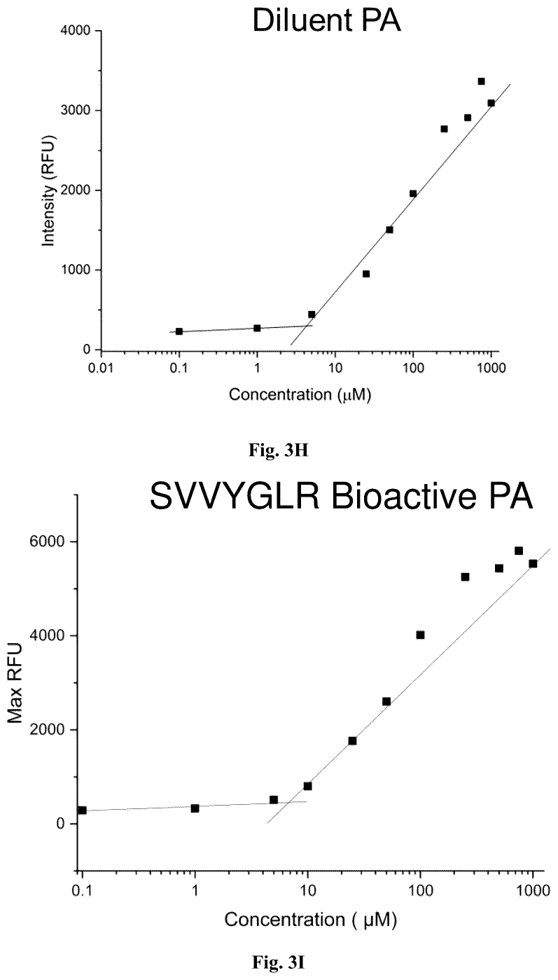

Peptide amphiphiles (PAs) are a class of biomaterial suitable for use in various applications that benefit from the provision of a scaffold. One such application is tissue regeneration. The peptide amphiphiles are biocompatible, biodegradable, and are able to mimic the scaffolding structure of an extracellular matrix using their filamentous supramolecular assembly.^{18,19} PA molecules. The scaffold is formed by employing interactions such as hydrophobic interactions, beta-sheet hydrogen bonding, and charge repulsion. This allows the peptide amphiphiles to rapidly self-assemble into elongated nanofibers (FIG. 3B). These nanofibers are shear thinning and able to rapidly self-heal following breakage allowing for, in the presence of ionic crosslinking, the ability to 3D-print self-supporting architectures,²⁰ or to employ spray delivery as demonstrated herein. Further, PA nanofibers are an ideal platform to incorporate bioactive peptide epitopes for inducing desired cell signaling and protein interactions, such as those that support the wound healing process.²¹⁻²³

Peptides as drugs and biologic mimetics have gained significant attention resulting from the combined advantages of structural stability and their lower cost of manufacturing afforded by the use of small molecules with the specificity, potency, and low toxicity of protein biologics.²⁴ At the same time, advances in peptide library screening such as phage display, proteomics, and structural biology have led to the discovery of short peptide sequences which can achieve the mimetic biological functions of the full-length proteins that inspired their design. With respect to tissue engineering, this approach has led to a number of growth factor and pro-survival peptide therapeutics being applied in regenerative applications.²⁴ A significant challenge however of peptide therapeutics is their generally poor pharmacokinetics resulting from rapid degradation, poor bioavailability, and metabolic clearance following delivery.²⁵

PA nanofibers provide a platform to achieve improved resistance to degradation, while also providing a means for localizing bioactivity at the site of injury, avoiding off target side effects.

The peptide amphiphiles (PA) of the present invention comprise an alkyl tail which allows for hydrophobic collapse and aggregation of molecules in an aqueous environment; hydrophobic peptide portion comprising one or more hydrophobic amino acids, a hydrophilic portion comprising one or more hydrophilic amino acids, for example, glutamic acid, to provide amphiphilicity and allow for molecular solubility; and an optional bioactive epitope, for example, a bioactive peptide. The plurality of peptide amphiphiles are non-covalently bonded together allowing for self-assembly and self-healing after shear thinning.

### The Hydrophobic Alkyl Tail Portion

The hydrophobic alkyl tail portion of the peptide amphiphile forms a core when assembled with a plurality of peptide amphiphiles into the supramolecular polymer nanofiber. The hydrophobic alkyl tail portion is coupled to a first end of the hydrophobic peptide portion. The hydrophobic alkyl tail portion allows for the hydrophobic collapse and aggregation of molecules when exposed to an aqueous environment, including biological buffers, salt solutions, biologically derived fluids, or any combination thereof.

The hydrophobic alkyl tail portion of the peptide amphiphile may be a linear or branched alkyl group. In a preferred embodiment, the alkyl tail is a linear alkyl group.

The alkyl tail may be an alkyl group derived from a saturated or unsaturated fatty acid having from about 6 to about 22 carbons atoms, or from about 8 to 20 carbon atoms, or from about 10 to 18 carbon atoms, or about 14 to 18 carbon atoms. For example, the hydrophobic alkyl tail may be derived from caproic acid, heptanoic acid, caprylic acid, nonanoic acid, capric acid, undecanoic acid, lauric acid, tridecanoic acid, myristic acid, palmitic acid, stearic acid, arachidic acid, behenic acid, and lignoceric acid.

### The Hydrophobic Peptide Portion

The hydrophobic peptide portion comprises one or more hydrophobic amino acid residues. The one or more hydrophobic amino acid residues are coupled between the hydrophobic alkyl tail portion and the hydrophilic portion. For example, the hydrophobic peptide portion of the peptide amphiphile may be derived from 1 to 10 hydrophobic amino acids, or from about 2 to 8 hydrophobic amino acids, or from about 3 to about 8 hydrophobic amino acids. The hydrophobic peptide portion may include, for example, one or more hydrophobic amino acid residues of amino acids selected from the group consisting of glycine, alanine, valine, leucine, phenylalanine, cysteine, isoleucine, and combinations thereof.

### The Beta-Sheet Forming Portion

The optional beta-sheet portion of the peptide amphiphile may form part of the hydrophobic peptide portion comprising one or more hydrophobic amino acid residues suitable for forming beta sheets. The beta sheet forming portion is an intermediate amino acid sequence of the peptide amphiphile molecule generally composed of three to ten amino acid residues with non-polar, uncharged side chains, selected for their propensity to form a beta-sheet secondary structure.

Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), Gly (G), (listed in order of their propensity to form beta sheets). However, non-naturally occurring amino acids of similar beta sheet forming propensity may also be used.

In a more preferred embodiment, a strong and a weak beta sheet former are used in combination, for example taking the form (X_{A})_{Na}, (X_{B})_{Nb}, (X_{C})_{Nc}, where X_{A} and X_{B} are selected from A. L. V. and G, X_{C} is any amino acid, Na and Nb are 2, 3 or 4 and Nc ranges from 0 to 3. An illustrative example is VVVAAAEEE (SEQ ID NO: 7) wherein. In a preferred embodiment, X_{C} is an amino acid residue with an amine-terminated side-chain, including but not limited to lysine or ornithine, wherein the amine functionality of said side-chain facilitates attachment of the lipophilic segment to the peptide.

### Hydrophilic Portion

The hydrophilic portion of the peptide amphiphile is included to provide solubility to the peptide amphiphile when in an aqueous environment. Also, the hydrophilic portion provides potential attachment sites to the peptide amphiphile for bioactive moieties. The hydrophilic portion may comprise from 1 to 12 hydrophilic amino acid residues, or from about 2 to 11 amino acid residues, or from about 3 to 10 amino acid residues.

For example, the hydrophilic portion may include hydrophilic amino acid residues derived from amino acids selected from serine, asparagine, glutamine, threonine, arginine, lysine, histidine, aspartic acid, and glutamic acid, or combinations thereof. Optionally, the hydrophilic portion may further include non-natural amino acids, and hydrophilic moieties without charge that can facilitate nanofiber self-assembly. When the hydrophilic portion comprises more than one hydrophilic amino acid residue, the hydrophilic amino acid residues may be the same or different.

### Unfunctionalized Peptide Amphiphile

In one embodiment, the peptide amphiphile without functionalization with a bioactive component may have the following Chemical Structure 1 (Diluent PA):

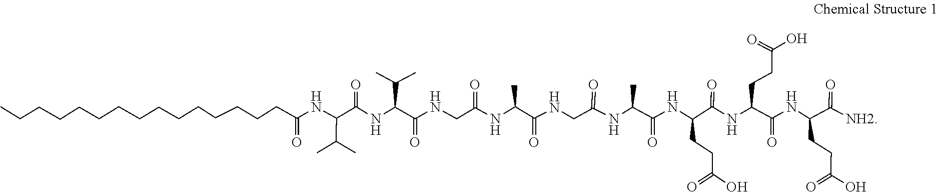 [SMILES: CCCCCCCCCCCCCCCC(=O)NC(C(=O)N[C@H](C(=O)NCC(=O)N[C@@H](C)C(=O)NCC(=O)N[C@@H](C)C(=O)N[C@H](CCC(=O)O)C(=O)N[C@@H](CCC(=O)O)C(=O)N[C@H](CCC(=O)O)C(N)=O)C(C)C)C(C)C]

### Bioactive Component

The bioactive component is positioned in the peptide amphiphile such that it is displayed on the periphery of the supramolecular polymer nanofibers when the plurality of peptide amphiphiles are assembled into the supramolecular polymer nanofiber matrix. This allows for cell interaction with the bioactive component. The bioactive component of the peptide amphiphile is coupled to the the hydrophilic portion of the peptide amphiphile such that the bioactive component is available for non-covalent interaction with other molecules or proteins.

The bioactive component may be selected from the group consisting of peptide sequence EMP1 (GGTYSCHFGPLTWVCKPQGG (SEQ ID NO.: 1)), ERK receptor activating REGRT (SEQ ID NO.2), SVVYGLR (SEQ ID NO.: 3) peptide sequence, QHREDGS (SEQ ID NO.4), cell adhesion peptide sequence, RGDS (SEQ ID NO: 5), bioactive wound healing peptide sequence, GLLSGINAEWPC (SEQ ID NO: 6), the antioxidant moiety lipoic acid, the antioxidant moiety protocatechuic acid (PCA), and the antioxidant moiety glutathione tripeptide. Antioxidants suitable for use to form the bioactive component may comprise one or more amino acids, wherein the antioxidant moiety is formed along at least one of the carboxyl group of the one or more amino acids.

For example, the bioactive component may have the following chemical structures:
EMP1—GGTYSCHFGPLTWVCKPQGG (SEQ ID NO.: 1), Chemical Structure 2,

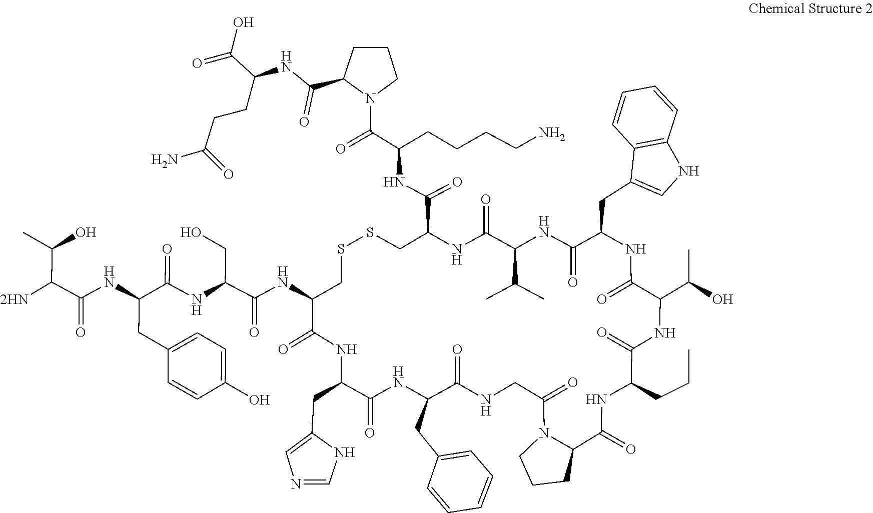

ERK receptor activating REGRT (SEQ ID NO.2), Chemical Structure 3,

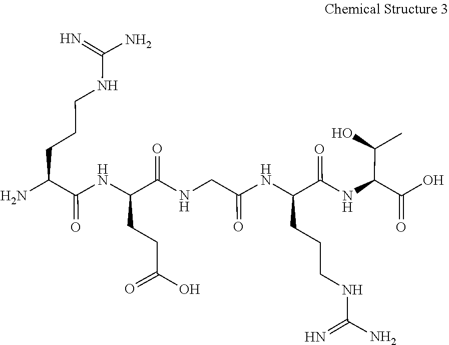 [SMILES: C[C@H](O)[C@H](NC(=O)[C@@H](CCCNC(=N)N)NC(=O)CNC(=O)[C@@H](CCC(=O)O)NC(=O)[C@@H](N)CCCNC(=N)N)C(=O)O]

SVVYGLR (SEQ ID NO.: 3), Chemical Structure 4,

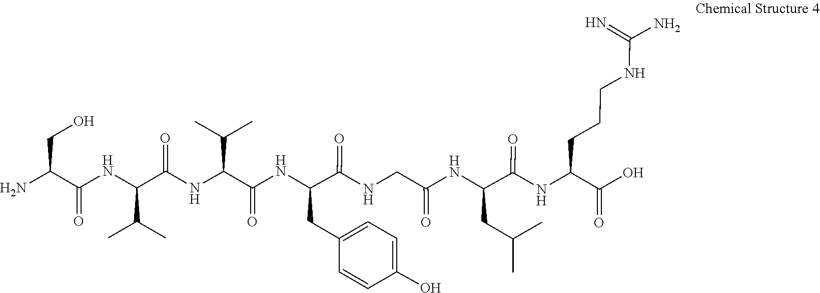 [SMILES: CC(C)C[C@@H](NC(=O)CNC(=O)[C@@H](Cc1ccc(O)cc1)NC(=O)[C@@H](NC(=O)[C@H](NC(=O)[C@@H](N)CO)C(C)C)C(C)C)C(=O)N[C@@H](CCCNC(=N)N)C(=O)O]

Peptide sequence, QHREDGS (SEQ ID NO.4), Chemical Structure 5,

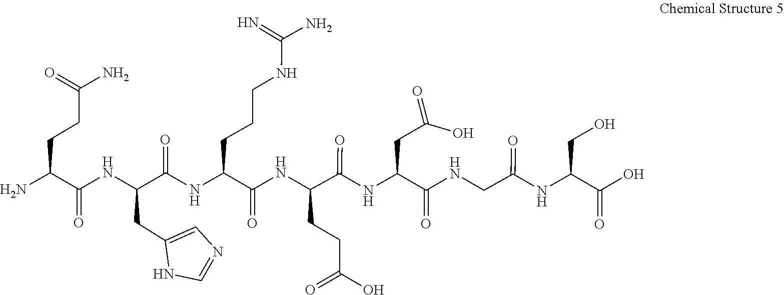 [SMILES: N=C(N)NCCC[C@H](NC(=O)[C@@H](Cc1cnc[nH]1)NC(=O)[C@@H](N)CCC(N)=O)C(=O)N[C@H](CCC(=O)O)C(=O)N[C@@H](CC(=O)O)C(=O)NCC(=O)N[C@@H](CO)C(=O)O]

Cell adhesion peptide sequence, RGDS (SEQ ID NO: 5), Chemical Structure 6,

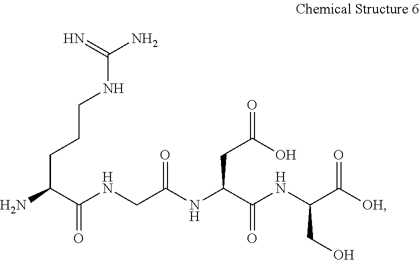 [SMILES: N=C(N)NCCC[C@H](N)C(=O)NCC(=O)N[C@@H](CC(=O)O)C(=O)N[C@H](CO)C(=O)O]

Bioactive wound healing peptide sequence, GLLSGINAEWPC (SEQ ID NO: 6), Chemical Structure 7,

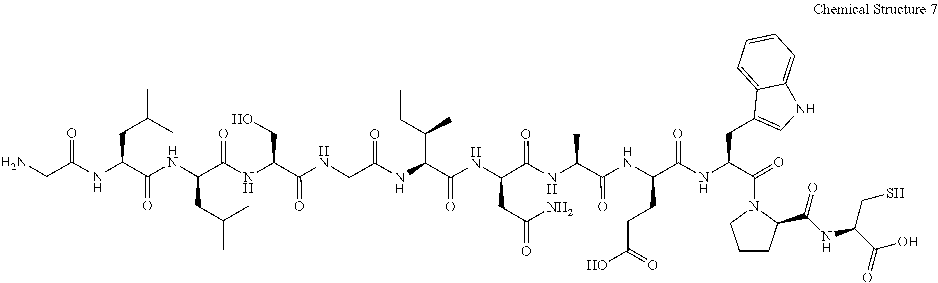 [SMILES: CC[C@@H](C)[C@H](NC(=O)CNC(=O)[C@H](CO)NC(=O)[C@@H](CC(C)C)NC(=O)[C@H](CC(C)C)NC(=O)CN)C(=O)N[C@H](CC(N)=O)C(=O)N[C@@H](C)C(=O)N[C@H](CCC(=O)O)C(=O)N[C@@H](Cc1c[nH]c2ccccc12)C(=O)N1CCC[C@@H]1C(=O)N[C@@H](CS)C(=O)O]

Antioxidant moiety of lipoic acid, Chemical Structure 8, wherein the moiety is formed along the carboxyl group of the lipoic acid,

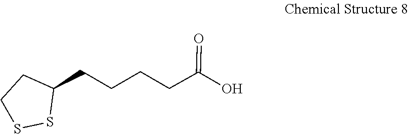 [SMILES: O=C(O)CCCC[C@@H]1CCSS1]

Antioxidant moiety of protocatechuic acid, Chemical Structure 9, wherein the moiety is formed along the carboxyl group of the protocatechuic acid,

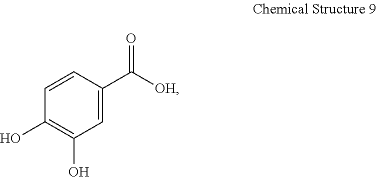 [SMILES: O=C(O)c1ccc(O)c(O)c1]

and
Antioxidant moiety glutathione tripeptide, Chemical Structure 10, wherein the moiety is formed along the carboxyl group of the glutathione tripeptide,

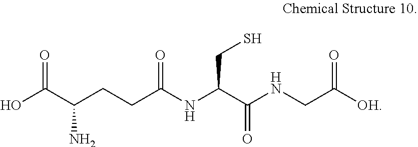 [SMILES: N[C@@H](CCC(=O)N[C@@H](CS)C(=O)NCC(=O)O)C(=O)O]

The peptide amphiphiles capped with a bioactive component of the present invention may have the following chemical structures, wherein V₃A₃E₃ (SEQ ID NO: 7) represents a beta sheet forming section:

Peptide Amphiphile Bioactive Epitope-Chemical Structure 11 (C₁₆-(SEQ ID NO: 8)) includes V₃A₃E₃ (SEQ ID NO: 7) and EMP1—GG-TYS(CHFGPLTWVCKPQGG)ₑ (SEQ ID NO.:1) where c represents cyclization of the sequence through the cysteine residues present within the parenthesis of SEQ ID NO.:1,

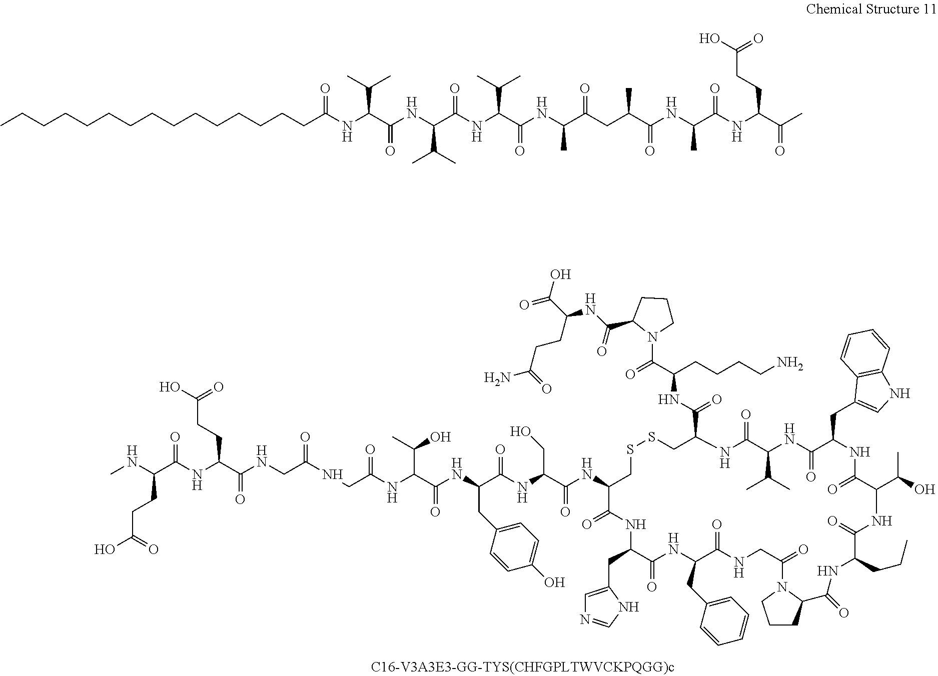

Peptide Amphiphile Bioactive Epitope-Chemical Structure 12 (C₁₆-(SEQ ID NO: 9)) includes V3A3E3 (SEQ ID NO: 7) linked by GGGG (SEQ ID NO: 10) to REGRT (SEQ ID NO.: 2),

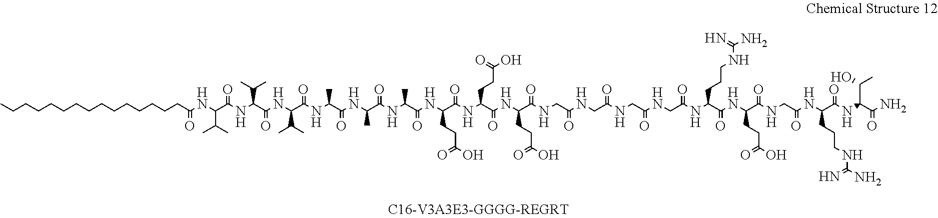 [SMILES: CCCCCCCCCCCCCCCC(=O)NC(C(=O)N[C@H](C(=O)N[C@@H](C(=O)N[C@@H](C)C(=O)N[C@H](C)C(=O)N[C@@H](C)C(=O)N[C@H](CCC(=O)O)C(=O)N[C@@H](CCC(=O)O)C(=O)N[C@H](CCC(=O)O)C(=O)NCC(=O)NCC(=O)NCC(=O)NCC(=O)N[C@@H](CCCNC(=N)N)C(=O)N[C@H](CCC(=O)O)C(=O)NCC(=O)N[C@H](CCCNC(=N)N)C(=O)N[C@H](C(N)=O)[C@@H](C)O)C(C)C)C(C)C)C(C)C]

Peptide Amphiphile Bioactive Epitope-Chemical Structure 13 (C16-(SEQ ID NO: 11)) includes V3A3E3 (SEQ ID NO: 7) linked by GG to SVVYGLR (SEQ ID NO: 3)

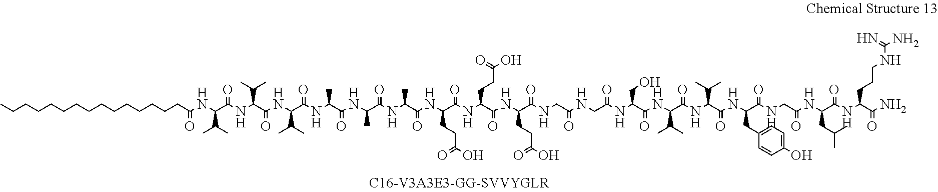 [SMILES: CCCCCCCCCCCCCCCC(=O)N[C@@H](C(=O)N[C@H](C(=O)N[C@@H](C(=O)N[C@@H](C)C(=O)N[C@H](C)C(=O)N[C@@H](C)C(=O)N[C@H](CCC(=O)O)C(=O)N[C@@H](CCC(=O)O)C(=O)N[C@H](CCC(=O)O)C(=O)NCC(=O)NCC(=O)N[C@@H](CO)C(=O)N[C@@H](C(=O)N[C@H](C(=O)N[C@H](Cc1ccc(O)cc1)C(=O)NCC(=O)N[C@H](CC(C)C)C(=O)N[C@@H](CCCNC(=N)N)C(N)=O)C(C)C)C(C)C)C(C)C)C(C)C)C(C)C]

Peptide Amphiphile Bioactive Epitope-Chemical Structure 14 (C₁₆-(SEQ ID NO: 12) includes V3A3E3 (SEQ ID NO: 7) linked by GG to QHREDGS (SEQ ID NO.: 4),

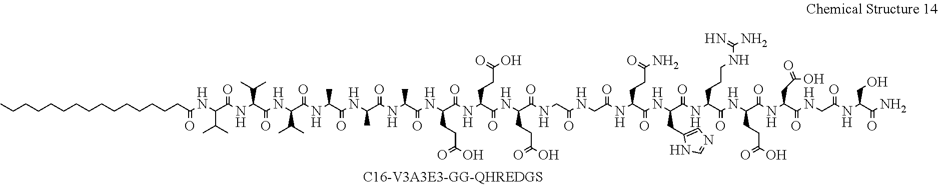 [SMILES: CCCCCCCCCCCCCCCC(=O)NC(C(=O)N[C@H](C(=O)N[C@@H](C(=O)N[C@@H](C)C(=O)N[C@H](C)C(=O)N[C@@H](C)C(=O)N[C@H](CCC(=O)O)C(=O)N[C@@H](CCC(=O)O)C(=O)N[C@H](CCC(=O)O)C(=O)NCC(=O)NCC(=O)N[C@@H](CCC(N)=O)C(=O)N[C@H](Cc1cnc[nH]1)C(=O)N[C@@H](CCCNC(=N)N)C(=O)N[C@H](CCC(=O)O)C(=O)N[C@@H](CC(=O)O)C(=O)NCC(=O)N[C@@H](CO)C(N)=O)C(C)C)C(C)C)C(C)C]

Peptide Amphiphile Bioactive Epitope-Chemical Structure 15 (C₁₆-(SEQ ID NO: 13)) includes V3A3E3 (SEQ ID NO: 7) linked by GGGG (SEQ ID NO: 10) to RGDS (SEQ ID NO.: 5),

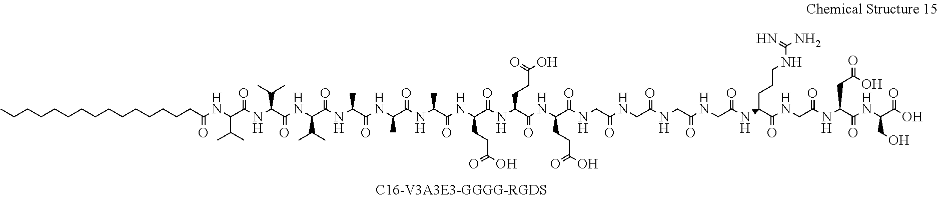 [SMILES: CCCCCCCCCCCCCCCC(=O)NC(C(=O)N[C@H](C(=O)N[C@@H](C(=O)N[C@@H](C)C(=O)N[C@H](C)C(=O)N[C@@H](C)C(=O)N[C@H](CCC(=O)O)C(=O)N[C@@H](CCC(=O)O)C(=O)N[C@H](CCC(=O)O)C(=O)NCC(=O)NCC(=O)NCC(=O)NCC(=O)N[C@@H](CCCNC(=N)N)C(=O)NCC(=O)N[C@@H](CC(=O)O)C(=O)N[C@H](CO)C(=O)O)C(C)C)C(C)C)C(C)C]

Peptide Amphiphile Bioactive Epitope—Chemical Structure 16 (C₁₆-(SEQ ID NO: 14)) includes V3A3E3 (SEQ ID NO: 7) linked by GG to GLLSGINAEWPC (SEQ ID NO.: 6),

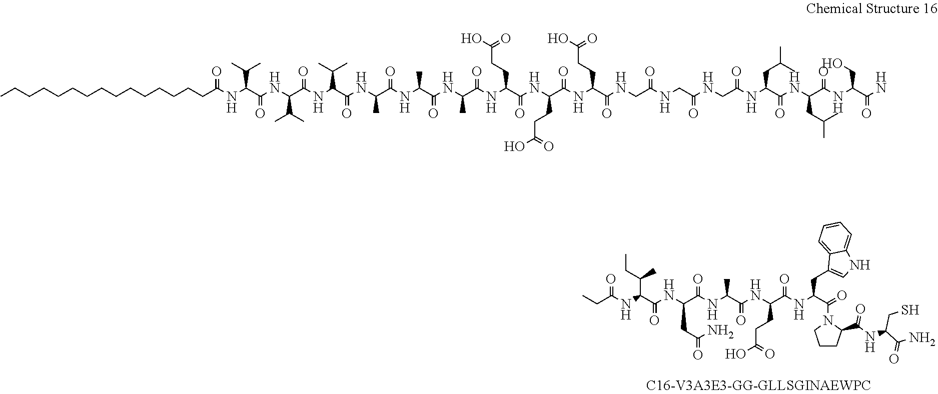

Peptide amphiphile with lipoic acid antioxidant moiety, Chemical Structure 17 (C₁₆-(SEQ ID NO: 15)-(LA) includes V3A3E3 (SEQ ID NO: 7) linked by lysine (K) to a lipoic acid (LA) antioxidant moiety. The lipoic acid antioxidant is attached with a gamma peptide linkage between the carboxyl group of the lipoic acid side chain and the amine of the lysine.

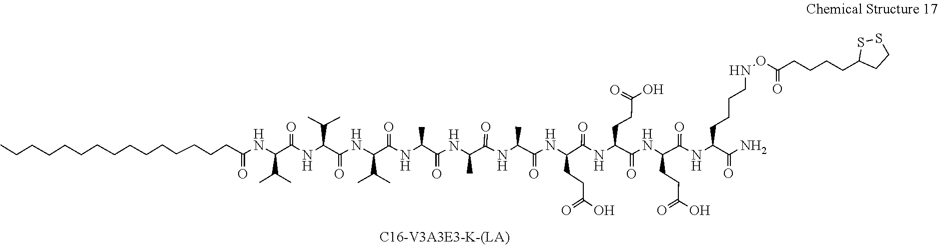 [SMILES: CCCCCCCCCCCCCCCC(=O)N[C@@H](C(=O)N[C@H](C(=O)N[C@@H](C(=O)N[C@@H](C)C(=O)N[C@H](C)C(=O)N[C@@H](C)C(=O)N[C@H](CCC(=O)O)C(=O)N[C@@H](CCC(=O)O)C(=O)N[C@H](CCC(=O)O)C(=O)N[C@@H](CCCCNOC(=O)CCCCC1CCSS1)C(N)=O)C(C)C)C(C)C)C(C)C]

Peptide amphiphile with protocatechuic acid (PCA), Chemical Structure 18 (C₁₆-(SEQ ID NO: 15)-PCA), includes V3A3E3 (SEQ ID NO: 7) linked by lysine (K) to a protocatechuic acid antioxidant moiety. The protocatechuic acid antioxidant is attached with a gamma linkage between the carboxyl group of the protocatechuic acid side chain and the amine of the lysine.

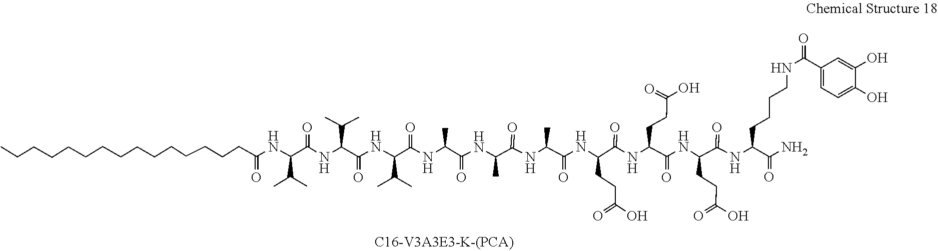 [SMILES: CCCCCCCCCCCCCCCC(=O)N[C@@H](C(=O)N[C@H](C(=O)N[C@@H](C(=O)N[C@@H](C)C(=O)N[C@H](C)C(=O)N[C@@H](C)C(=O)N[C@H](CCC(=O)O)C(=O)N[C@@H](CCC(=O)O)C(=O)N[C@H](CCC(=O)O)C(=O)N[C@@H](CCCCNC(=O)c1ccc(O)c(O)c1)C(N)=O)C(C)C)C(C)C)C(C)C]

Peptide amphiphile with glutathione tripeptide antioxidant moiety, Chemical Structure 19 (C₁₆-(SEQ ID NO: 16)), includes V3A3E3 (SEQ ID NO: 7). The carboxyl group of the cysteine residue is attached by normal peptide linkage to glycine,

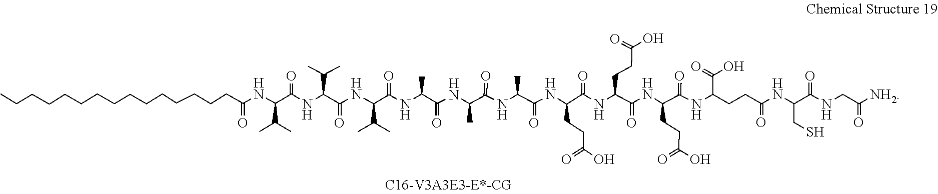 [SMILES: CCCCCCCCCCCCCCCC(=O)N[C@@H](C(=O)N[C@H](C(=O)N[C@@H](C(=O)N[C@@H](C)C(=O)N[C@H](C)C(=O)N[C@@H](C)C(=O)N[C@H](CCC(=O)O)C(=O)N[C@@H](CCC(=O)O)C(=O)N[C@H](CCC(=O)O)C(=O)NC(CCC(=O)NC(CS)C(=O)NCC(N)=O)C(=O)O)C(C)C)C(C)C)C(C)C]

The pro-regenerative and bioactive PA molecules depicted above are designed for enhanced cell migration to the wound site and wound healing capability following spray delivery and application to a burn injury (FIG. 3a).

The presentation of bioactive peptide sequences, to localize and mimic the activity of growth factors and important cell signaling groups on a PA nanofiber scaffold, can be used to overcome challenges associated with the expense, poor half-life, and off target effects of known recombinant protein and peptide therapies. The PA with the bioactive component, i.e., bioactive epitope SVVYGLR (SEQ ID NO.: 3) combines bioactive peptide sequence SVVYGLR of SEQ ID NO.: 3 derived from the protein osteopontin, originally discovered in bone that has shown pro-regenerative capabilities in a number of tissues, including skin.²⁶ This peptide sequence has been effective in promoting dermal wound healing in a rat full thickness excision model through enhanced fibroblast migration and differentiation.²⁷

Erythropoietin (EPO) is a potent cytokine with great potential in skin wound healing because it stimulates all phases of the wound healing process, protects ischemic tissues, and promotes tissue regeneration. EPO acts on nonhematopoietic cells where it promotes cellular proliferation and differentiation, it is cytoprotective, and a powerful inhibitor of apoptosis.²⁸ Further, a number of studies have demonstrated that EPO is therapeutically beneficial when it is used to treat acute and chronic skin wounds in vivo.^{28, 29} However, despite these promising pre-clinical studies, EPO has not been translated into clinical practice in the treatment of wounds, most likely because of the challenges associated with protein therapies namely, cost, short half-life, the need for repeated dosing regimens, and the potential for immunogenicity or off target effects.³⁰ Significant opportunities exist in the presentation of bioactive peptide sequences, to both localize and mimic the activity of EPO on a PA nanofiber scaffold, overcoming challenges associated with the expense, poor half-life, and off target effects associated with recombinant protein therapies.

The peptide sequence, EMP1 (GGTYSCHFGPLTWVCKPQGG (SEQ ID NO.: 1)), cyclized through the cysteine residues, was discovered to be a potent mimic of EPO, capable of activating the receptor of EPO as well as stimulating erythropoiesis in mice.³¹ Presenting this cyclized sequence on the surface of a PA nanofiber provides the opportunity for multivalent receptor interactions resulting from the biomimetic epitope presentation being non-covalently polymerized within the PA nanofiber assembly.

Similarly, the ERK/MAPK pathway is integral in the cell signaling of migratory cellular responses. Re-epithelization, the process of epithelial cells migrating across a wound to re-establish the damaged epidermis, is an essential early process in wound healing.³² Unsuccessful re-epithelization can lead to persistent infection, chronic wounds, and poor scar outcomes.^{32,33} As a result, timely acute wound healing and effective cell migration remain as paramount considerations in the regeneration of skin following a burn injury. The PA incorporating QHREDGS (SEQ ID NO.: 4), a pro-survival peptide derived from angiopoietin, was recently demonstrated to significantly accelerate wound closure in a diabetic wound model through enhanced keratinocyte survival and migration.³⁴

Similarly, the short pentapeptide (REGRT (SEQ ID NO: 2) has demonstrated the promotion of wound healing in rodent models of both full thickness injury,³⁵ and atopic dermatitis.³⁶ The success of this peptide in wound healing was a direct result of effective phosphorylation of the ERK receptor which in turn promoted migration of both keratinocytes and fibroblasts at the injury site. The PA with the bioactive epitope REGRT (SEQ ID NO.: 2), incorporates this peptide sequence to activate the ERK/MAPK signaling pathway to promote cellular migration following injury, ultimately driving keratinocyte migration and enhanced wound closure.

These PA molecules will be assembled into bioactive PA nanofibers to create a therapeutic scaffold suitable for promoting rapid skin regeneration following injury (FIG. 3b). Further, due to the non-covalent interactions between PA molecules, these assemblies self-heal following high shear events such as injection,^{37, 38} 3D bioprinting,²² or, as demonstrated herein, through spray delivery in the presence of cells (FIG. 3c). Herein we have confirmed the potential for spray delivery of PA nanofibers to reform a scaffold that mimics the extracellular matrix following spray delivery, while maintaining high cell viability (FIG. 3d). The PA nanofibers described herein are an ideal target to combine with cells, spray delivery onto a burn injury, and self-assembly into a scaffold following spraying to support skin regeneration and localize the cells and bioactive epitopes. Further, the ability to undergo ionic crosslinking with physiological media will support rapid scaffold formation, preventing cell run off at the site of injury, and enhancing the opportunity for localized healing (FIG. 3e).

### Biological Material

The plurality of peptide amphiphiles may be combined with a biological material and/or a buffer for delivery to a subject.

The biological material to be administered may include biological fluids, cells, and/or tissue material. In some embodiments, one or more of the biological materials administered may be synthetic. In other embodiments, the biological materials may be obtained from a donor. The biological material may also be autologous cells (obtained from the recipient subject).

The compositions of the present invention enable rapid cell spray preparation, i.e. in as little as 30 minutes to 4 hours, including the steps of collection of a skin sample, matrix digestion, and spray delivery. Furthermore, the sprayable compositions of the present invention, when including autologous cells, result in a significant reduction in the required amount of donor skin for wound treatment when compared to existing systems which may also require days or weeks to grow adequate skin cells.

For example, the method for promoting skin cell tissue regeneration may include steps of:
preparing the composition as discussed above,
collecting a biological material,
mixing a buffer and the biological material with the prepared composition to produce a suspension or solution of the composition, and
delivering the suspension or solution to a patient, wherein the step of delivery is carried out using high shear conditions.

In an embodiment, the biological material may comprise autologous skin cells. In this embodiment, the step of mixing the biological autologous skin cells with the composition to produce a suspension or solution can be carried out rapidly, allowing a physician to collect the autologous skin cells from the patient and treat the patient within a short period of time. For example, the biological autologous skin cells may be mixed with the composition to produce a suspension or solution during a time period having a duration of from about 15 minutes to 2 hours, or from about 15 minutes to 1 hours, or from about 15 minutes to about 45 minutes, or from about 20 minutes to about 40 minutes, or from about 30 minutes, or less than 2 hours, or less than 1 hour.

### EXAMPLES

### Example 1—Synthesis, Characterization, and Assessment of Bioactive PA Nanofibers Designed to Encourage Skin Regeneration Following Burn Injury

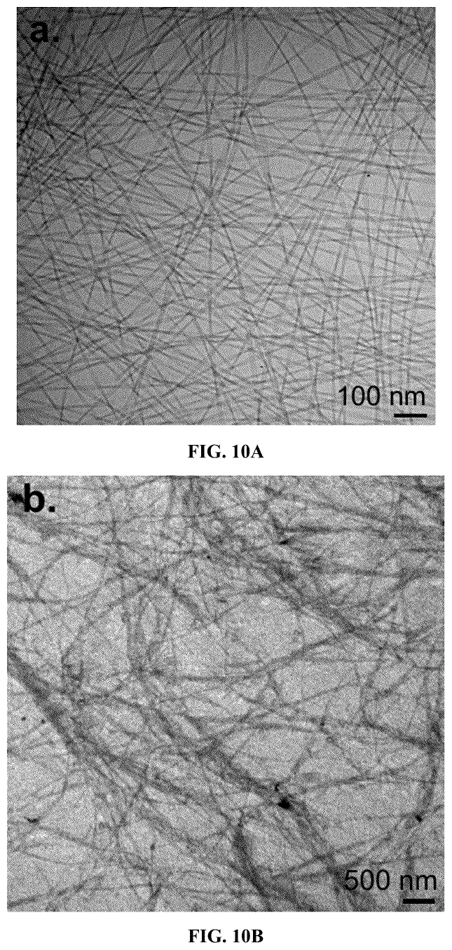

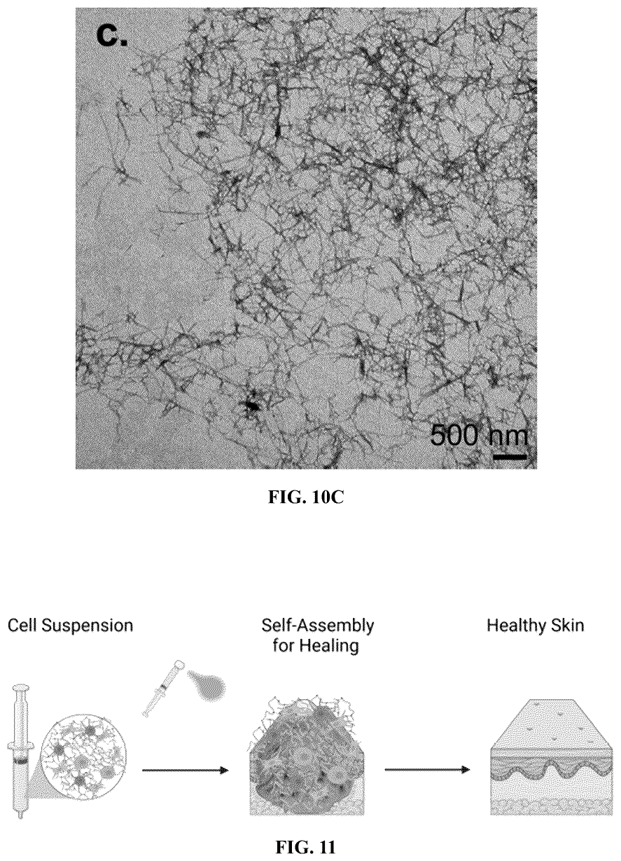

The PA molecules are synthesized using standard solid phase peptide synthesis methods, purified to >95% purity by high-performance liquid chromatography (HPLC), and confirmed by liquid chromatography mass spectrometry (LC-MS) as described previously.²² The PA molecules were designed to contain bioactive peptide groups to promote skin cell migration and proliferation following injury (FIG. 3a). The bioactive PA molecules can be co-assembled with a non-bioactive PA (diluent PA, FIG. 3a) or a fluorescently labelled variant to enhance fiber formation and the ability to fluorescently track these assemblies respectively (FIG. 3b). PA nanofiber self-assembly and morphology can be assessed by transmission electron microscopy (TEM) (FIGS. 10A-10C), and small angle X-ray scattering (SAXS), while the hydrogen bonding between molecules within the nanofibers can be confirmed by circular dichroism (FIG. 3g), and wide-angle X-ray scattering (WAXS). These experiments allow us to understand both fiber formation of the molecules as well as the strength of interaction holding the PA molecules together in the assemblies. The critical aggregation concentration, a measure of the propensity for the molecules to assemble together, was assessed using a Nile red spectrophotometric assay as known in the art (FIGS. 3H-3I). The ratio of molecules within the co-assembly can be systematically modified of each bioactive PA molecule within the nanofiber assemblies without disrupting fiber formation. This ensured that the physical extracellular matrix mimetic architecture of these biomaterials was maintained. A typical co-assembly of molecules is: 20% of each bioactive PA (red, FIG. 3b), 80% diluent fiber forming PA (grey, FIG. 3b).

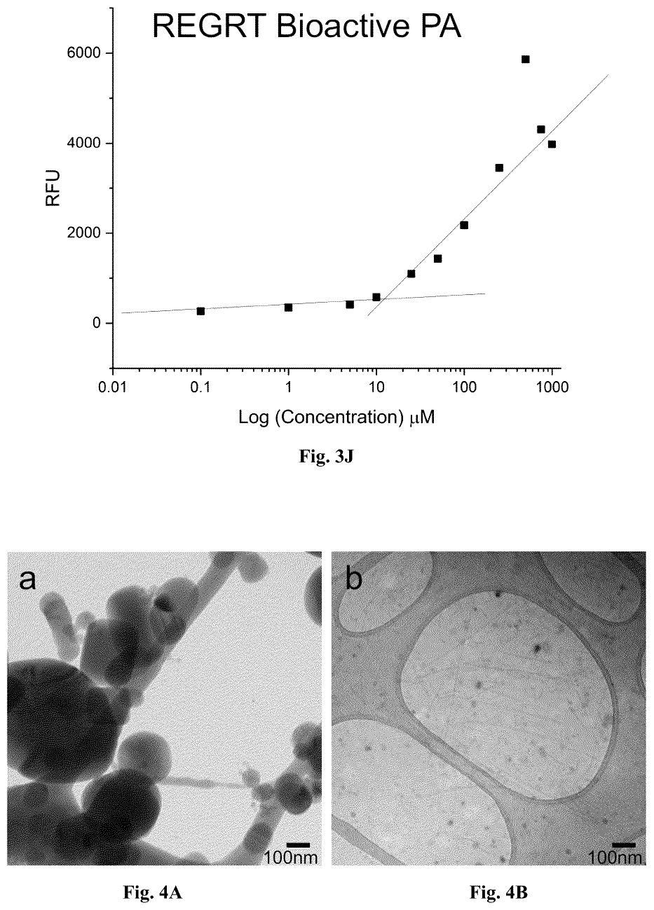

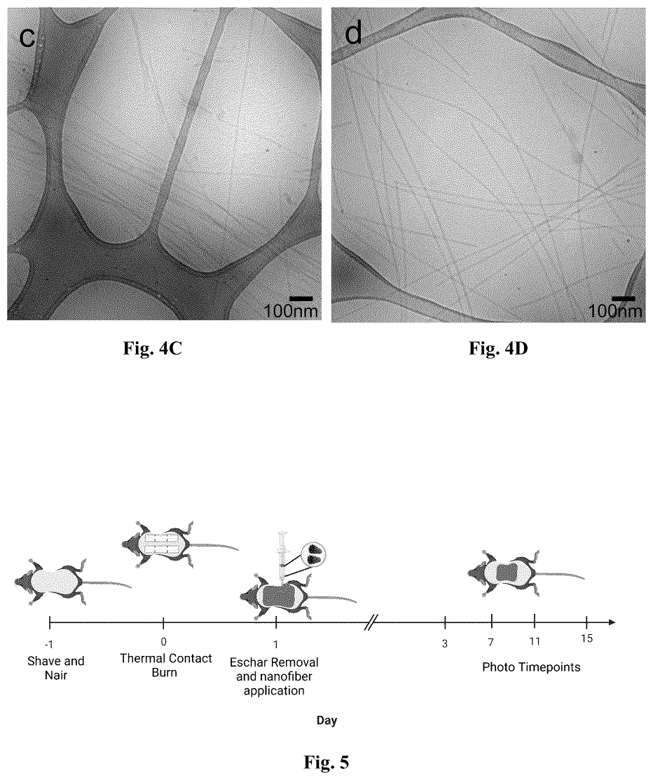

The architecture and self-healing capabilities of the co-assembled bioactive PA nanofibers were characterized following spray delivery. A unique feature of PA nanofibers is the non-covalent interactions between molecules, which results in rapid and programmed self-assembly of these molecules into elongated nanofibers (FIG. 4). Further, these non-covalent interactions can be reversibly broken and reformed, allowing these nanofibers to self-heal following physical stresses such as ultrasonication or the high shear associated with injection or spray delivery. As a result of these characteristics, PA nanofibers present as an ideal material for spray delivery, in the presence of cells, through providing an environment reminiscent of the lost extracellular matrix to support skin regeneration.

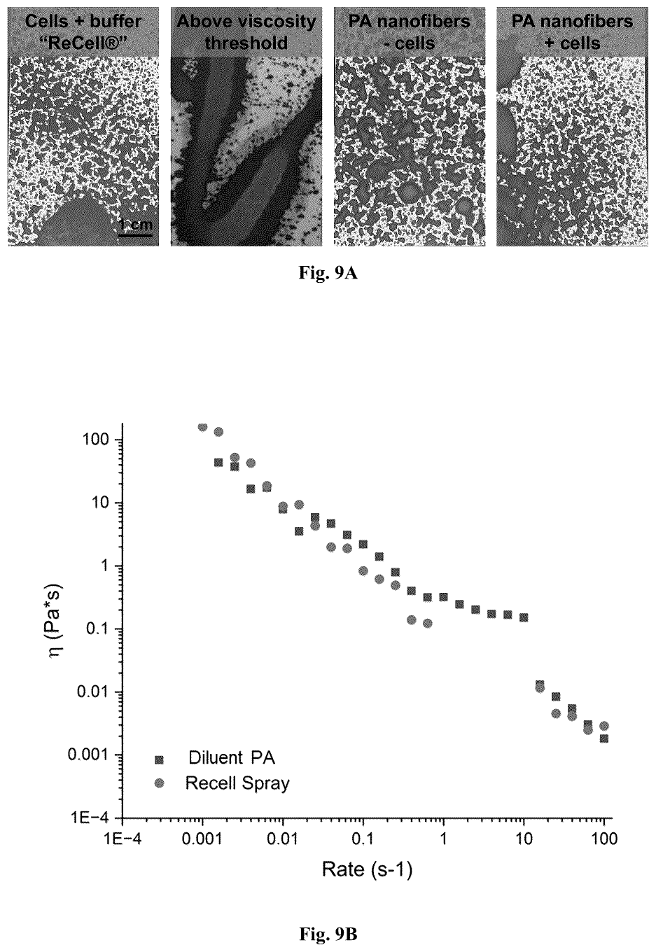

The initial tests utilized spray nozzles from the ReCell® system. The PA nanofibers were prepared at a range of concentrations and viscosities by rheology. To obtain complex viscosity η (Pa*s), a steady rate sweep test on the rheometer was performed using the cone-plate geometry. Sample was pipetted onto the bottom plate and the plates were brought together, ensuring sufficient sample loading by the visualization of concave out solution. Measurements were obtained over a set frequency range of from 0.0001 s⁻¹ to 100 s⁻¹ and the resulting viscosity was measured (FIG. 9b).

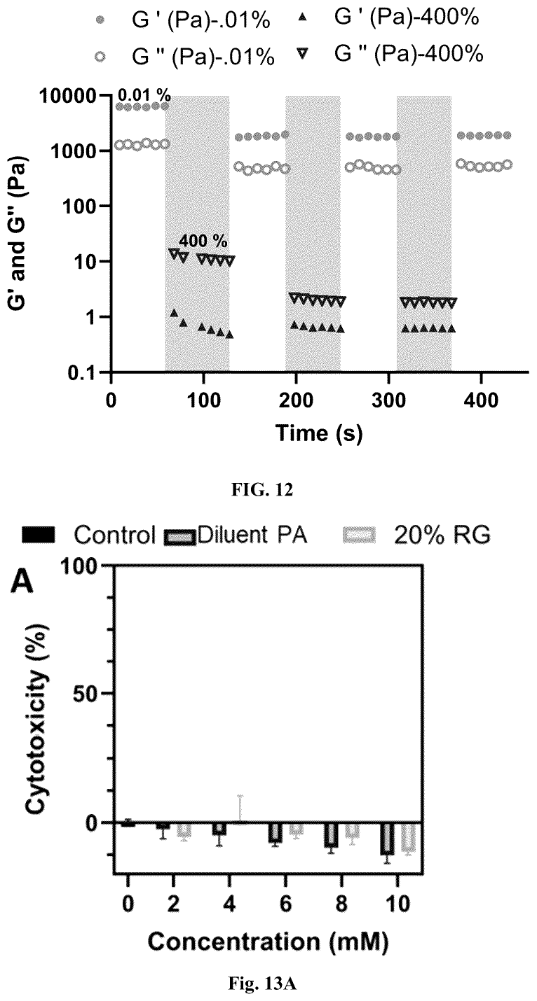

To determine the propensity for these materials to self-heal following deformation which would occur during a spray application, a six-step oscillatory strain test was utilized. Samples were ionically gelled on the bottom plate, and time-dependent sweeps were applied with alternating high-low strain steps which allowed the monitoring of material recovery. The low strain is selected where elastic behavior dominates, and the high strain is selected outside of the linear viscoelastic region for the material where complete material deformation occurs. By returning to low strain rate, the elastic modulus recovery and thus self-healing ability can be evaluated. The diluent PA exhibited near complete property recovery (FIG. 12).

Following surgical protocols and using a water sensitive card as a substrate (SPOTONR™), the spray coverage of the bioactive PA nanofibers was assessed relative to concentration. Parameters such as solution concentration, viscosity, rate of discharge, droplet density, area covered, and dose administered were systematically investigated and assessed using image analysis software (FiJi, NIH, Bethesda, MD) to determine optimal spraying parameters (FIG. 9a). Importantly, at the appropriate viscosity of PA nanofibers in the presence of cells and buffer, the spray deposition pattern of the ReCell® device with cells alone could be accurately recreated in the presence of the bioactive scaffold. Finally, scanning electron microscopy was used to investigate the formation of an interconnected bioactive PA nanofiber scaffold following spray delivery on the cellular length scale, and the images were compared to syringe deposited PA nanofibers as an appropriate control (FIG. 3c).

The spray delivery of the bioactive PA nanofibers resulted in an interconnected scaffold, similar to the extracellular matrix. Alternatively, the bioactive PA nanofibers may be delivered via pipette delivery (or extrusion through a needle), both of which were also shown to maintain nanofiber architecture, and these delivery methods may be utilized for downstream experiments with cells subsequently sprayed onto this bioactive scaffold.

### Example 2—Examination in Vitro Pro-Regenerative Capabilities of the Bioactive PA Nanofiber Scaffolds Following Spray Delivery

The cell survival was investigated following spray delivery in the presence of the bioactive PA nanofiber scaffold. The initial screening studies involved human embryonic kidney cells (HEK293) for high throughput screening and optimization of processing conditions. Initial screening of bioactive PA nanofibers using a lactate dehydrogenase (LDH) assay demonstrated low cytotoxicity of the bioactive PA nanofiber scaffolds across a broad range of concentrations (FIG. 3D). Cells were detached with trypsin and then suspended in cell media and Ringer's lactate solution to mimic the processing conditions of the ReCell® system. These suspensions were mixed with a concentrated solution of the bioactive scaffold before spraying the scaffold into culture dishes. Changes in cellular metabolic activity were analyzed using the Vybrant MTT assay (Life Technologies) and cell viability was assessed using a calcein and propidium iodide Live/Dead™ assay (ThermoFisher), followed by fluorescent imaging and quantification.

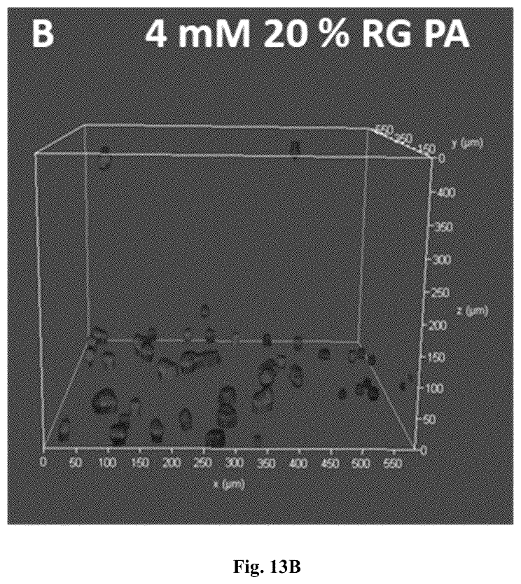

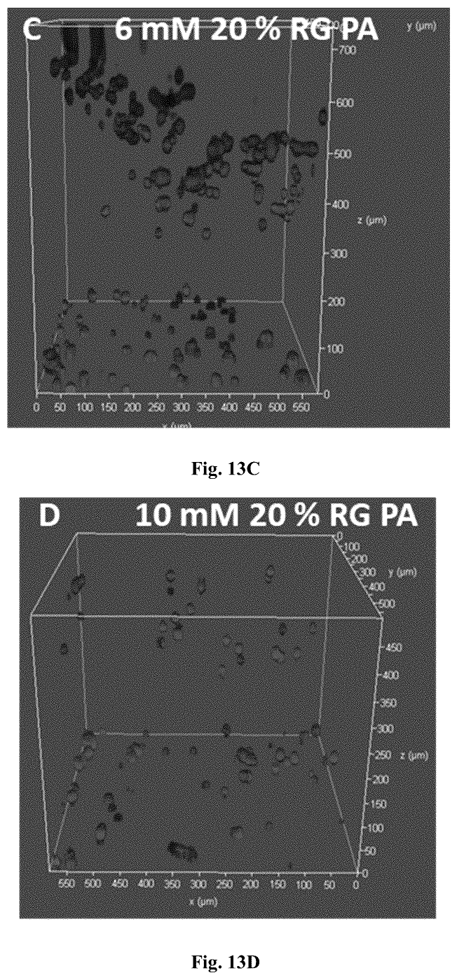

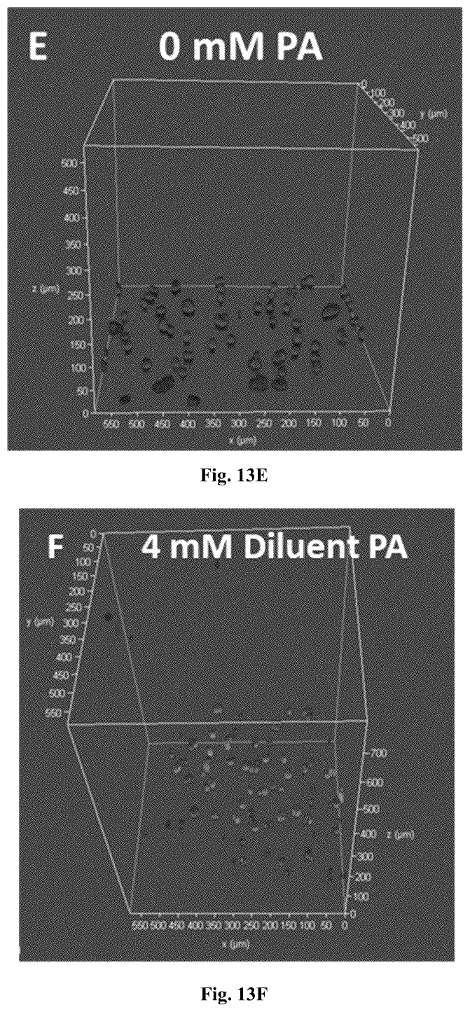

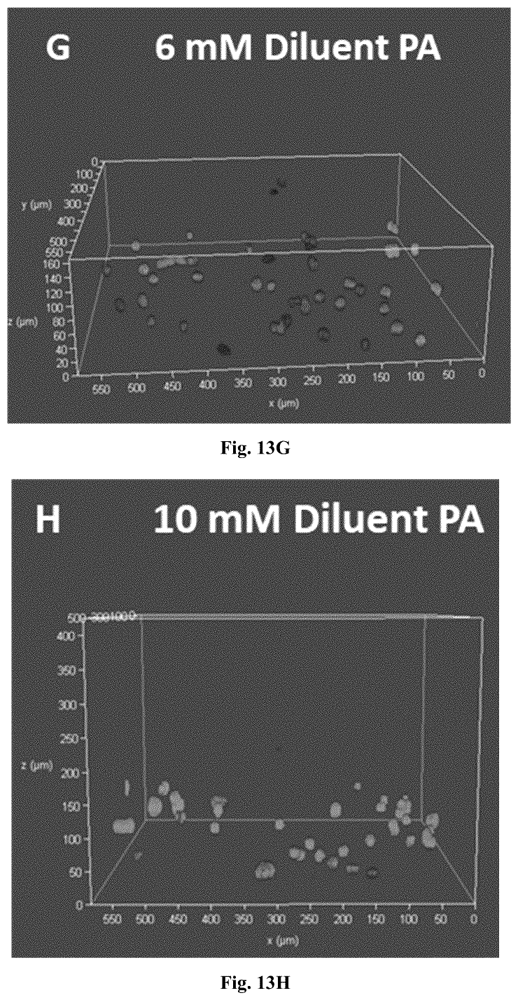

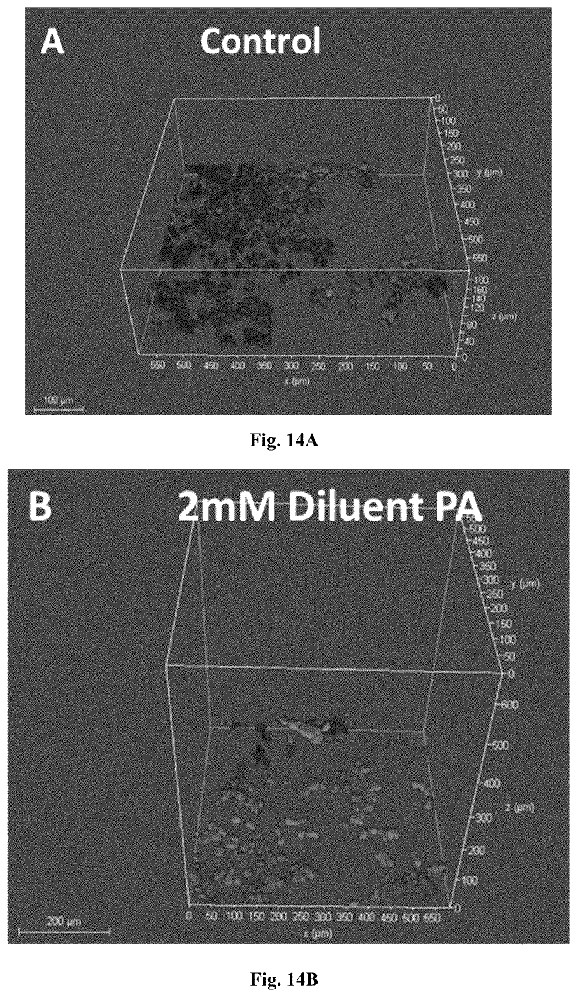

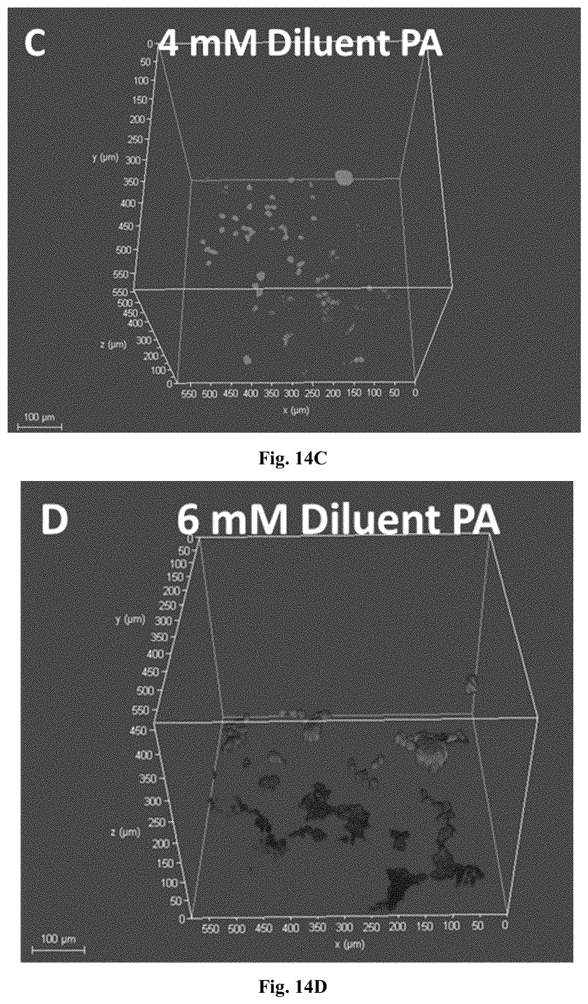

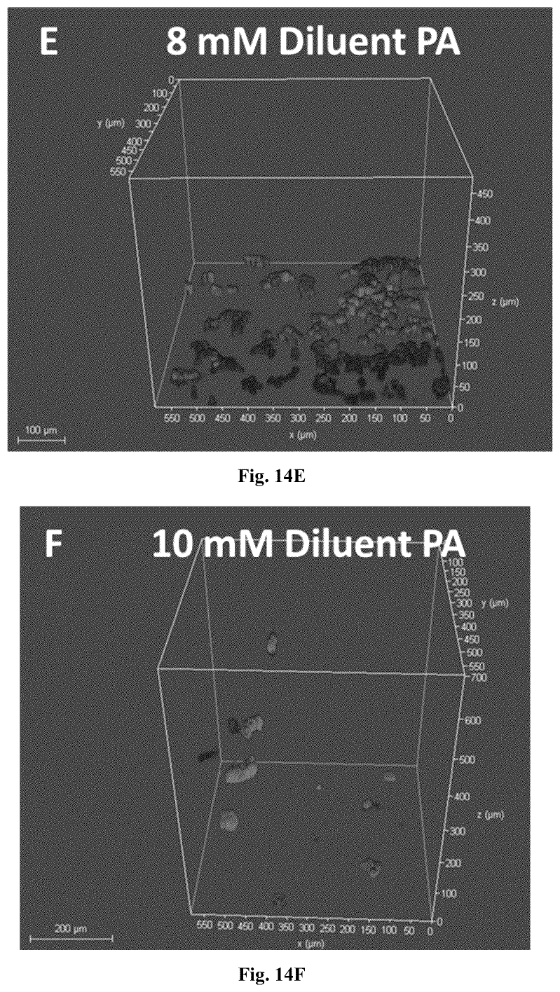

The cytotoxicity of high-concentration PAs was evaluated with a lactate dehydrogenase (LDH) assay paired with live imaging of cells in the PA matrix using confocal microscopy at 24 hours (FIG. 13). This highlighted cells that were healthy and capable of migrating through the matrix over a range of PA concentrations with no adverse effects. There were no observable differences between the diluent PA and the bioactive material (20% REGRT). To further investigate cell migration and viability, cells were stained with Calcein-am and imaged after 72 hours, where PAs were initially gelled with cells seeded on top and, as shown by imaging, cells had almost completely migrated through the matrix (FIG. 14).

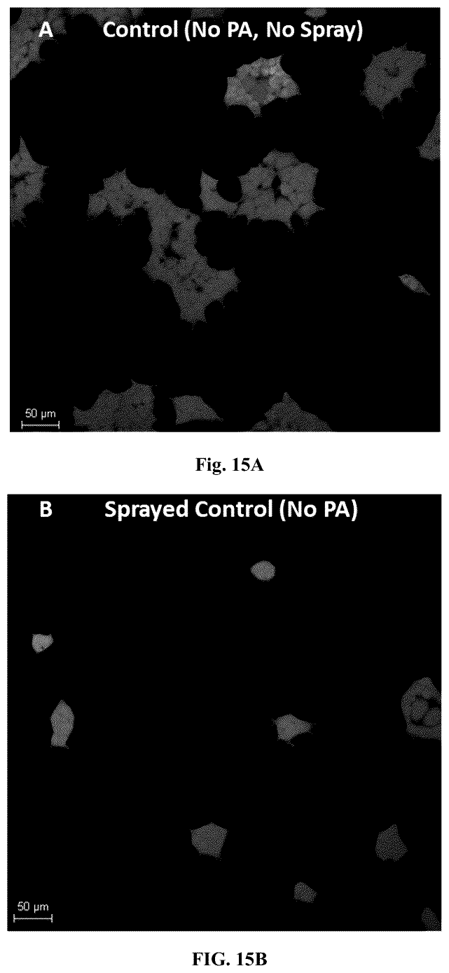

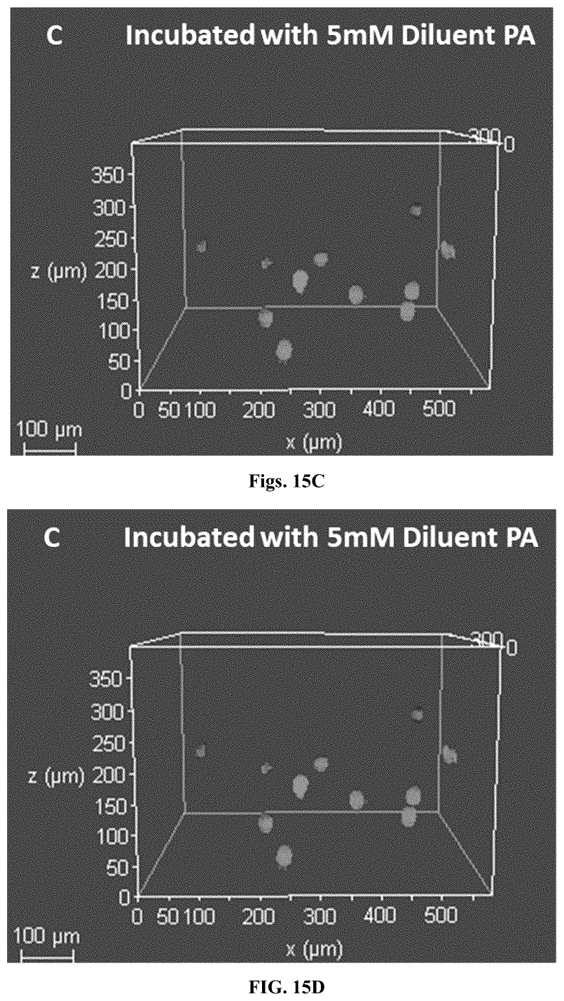

Finally, to evaluate the ability to spray these gelled materials with cells, a solution of cells in Ringers Lactate Solution was combined with a diluent PA stock solution to achieve a concentration of 5 mM PA and 20 mM CaCl₂) and sprayed using the ReCell® nozzle. Cells not sprayed, sprayed without any PA, incubated with diluent PA but not sprayed, and cells sprayed with diluent PA were then plated and left for 48 hours. They were then visualized using Calcein-am, a stain that is only fluorescent in live cells, on a confocal microscope (FIG. 15). Taken together, these analyses determined the biocompatibility of the scaffolds, the cell survival throughout the spraying process, and the proliferative potential of cells within the scaffolds. The results were compared to appropriate controls consisting of cells cultured in plates (without spray delivery) and cells sprayed without the presence of the bioactive scaffold.

### Example 3A—Assessment of the Therapeutic Efficacy of the Bioactive PA Nanofiber Scaffold Sprayable Therapy in a Burn Injury Murine Model

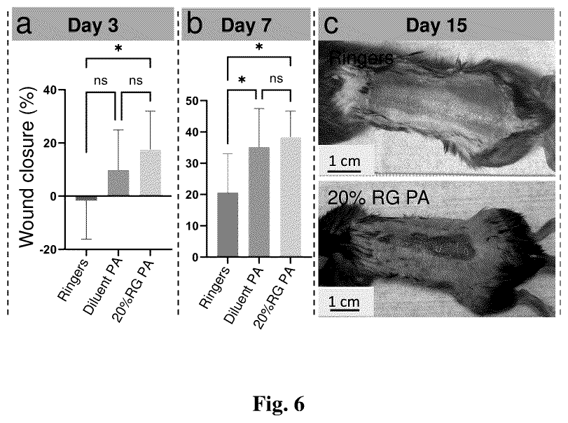

The benefits of, and potential for, regeneration of the biocompatible and bioactive PA nanofiber scaffold was assessed in a mouse burn injury model. This example demonstrated enhanced kinetics of wound closure in the presence of the bioactive PA nanofibers. (See FIGS. 5 and 6). A 1×1 cm diameter steel billet was heated to 100° C. in a dry heat bath. Mice were anesthetized using isoflurane throughout the procedures. After successful anesthesia had been achieved, mice were clipped circumferentially on their trunks/dorsum and prepped with betadine. Burn injury was accomplished by applying the previously heated billet to the dorsum of each animal for 10 seconds. A total of 6 applications of the rod (3 on each side of the midline back) produced an approx. 20% total body surface area (TBSA) full thickness burn. Mice were further wrapped with a self-adherent wrap to protect the wound. Mice were resuscitated using 1.0 mL i.p. saline (which included 0.03 mL Buprenex SR and 0.06 mL Meloxicam SR) and placed onto warming pads until full recovery from anesthesia was determined. 8 hours post-burn, they received a 2nd bolus of 1 mL i.p. fluid. 24 hrs post-burn injury, the burn wound eschar full thickness was removed with a scalpel to allow the peptide amphiphile nanofibers to be absorbed. This debridement correlates with human burn wound debridement in human burn injury care/treatment. The mice again were under anesthesia (Isoflurane) and analgesia (Buprenex SR and Meloxicam SR) during this procedure and then were provided analgesia (Buprenex SR) within a 24 hr period after the procedure to ensure no waning of pain relief was experienced post-operation. Bioactive peptide amphiphile nanofibers were topically applied directly following burn injury at a concentration of 10 mM prepared in sterile Lactated Ringer's Solution supplemented with calcium. Healing was assessed following a single application. Peptide amphiphile nanofibers were topically applied to injuries via extrusion through a syringe directly onto the injury site. Treatments were applied including a Ringers buffer control (Ringers), the diluent PA (diluent PA, PA nanofiber control), and a bioactive PA nanofiber (20% RG PA). An initial concentration of 20% of the bioactive PA in the co-assembly was chosen. The inclusion of the RG PA bioactive nanofibers significantly accelerated wound closure relative to controls at days 3 and 7 post treatment (FIGS. 6A and 6B), with wound closure at the conclusion of the study (day 15) appearing to be markedly improved (FIG. 6C). Quantification of wound healing was accomplished by digital image planimetry with ImageJ at 3 days, and 7 days post treatment, with representative images of wound healing at day 15 post treatment also being provided. 20% RG PA displays accelerated wound healing in early time points, with diluent PA becoming significant at 7 days. Data displayed as mean±s.d. with significance assessed by one-way ANOVA and post hoc Tukey Test, *p<0.05.

### Example 3B—Assessment of the Therapeutic Efficacy of the Bioactive PA Nanofiber Scaffold Sprayable Therapy in a Pre-Clinical Porcine Full-Thickness Excision Model

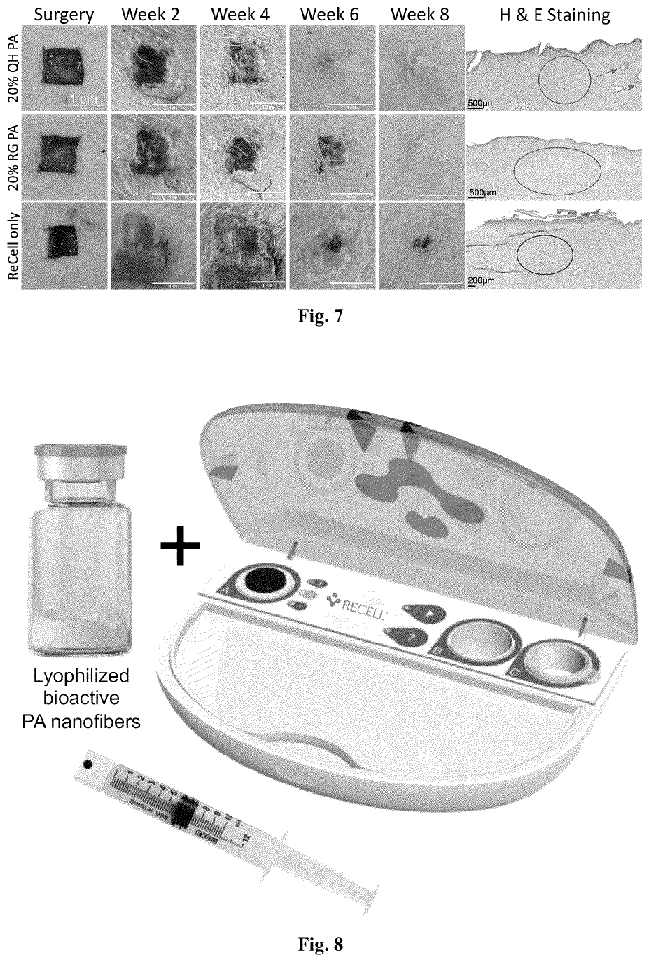

Following promising murine results with the scaffolds alone, a pilot porcine full thickness excision model of wound healing was used to assess efficacy of the bioactive PA nanofibers with ReCell® autologous skin cell suspension (ASCS) delivery. Full thickness excision injuries (1 cm×1 cm) were created, with harvested skin utilized for ASCS preparation following ReCell® clinical protocols. This cell suspension was combined with the bioactive PA nanofibers before being applied to the wounds following the ReCell® clinical protocol. Over the 8-week study wounds appeared to heal faster in the presence of the bioactive PA nanofibers and also showed evidence of regenerated extracellular matrix architecture more analogous to healthy skin than to scar tissue formation (FIG. 7). Additional analysis of cell populations following treatment by both flow cytometry and immunohistochemistry on fresh-frozen samples also indicated positive improvements in wound healing progression in the presence of the bioactive PA nanofibers.

FIG. 7 provides photographs of wound healing over the 8-week study demonstrating a trend towards faster wound healing for the 20% QH and 20% RG bioactive PA treated samples relative to ReCell® only control. Histology assessments with thick and aligned deposited ECM (red circle) observed in ReCell® only treated samples, compared to regions of random ‘basket weave’ architecture of ECM (green circles) and evidence of immature hair follicles (green arrows) in bioactive PA nanofiber treated samples all suggest improved skin architecture and enhanced healing outcomes.

### REFERENCES

All references cited herein are hereby incorporated by reference in their entirety as if set forth herein.
(1) Dunkin, C. S. J.; Pleat, J. M.; Gillespie, P. H.; Tyler, M. P. H.; Roberts, A. H. N.; McGrouther, D. A. Scarring occurs at a critical depth of skin injury: Precise measurement in a graduated dermal scratch in human volunteers. Plast Reconstr Surg 2007, 119 (6), 1722-1732. DOI: 10.1097/01.prs.0000258829.07399.f0.
(2) ReCell—Cutting edge technology for skin restoration. Avita Medical, 2021. https://avitamedical.com/product/ (accessed 2021.
(3) Larson, K. W.; Austin, C. L.; Thompson, S. J. Treatment of a Full-Thickness Burn Injury With NovoSorb Biodegradable Temporizing Matrix and RECELL Autologous Skin Cell Suspension: A Case Series. J Burn Care Res 2020, 41 (1), 215-219. DOI: 10.1093/jbcr/irz179.
(4) Hayes, P. D.; Harding, K. G.; Johnson, S. M.; McCollum, C.; Teot, L.; Mercer, K.; Russell, D. A pilot multi-centre prospective randomised controlled trial of RECELL for the treatment of venous leg ulcers. Int Wound J 2020, 17 (3), 742-752. DOI: 10.1111/iwj.13293.
(5) Holmes, J. H. t.; Molnar, J. A.; Shupp, J. W.; Hickerson, W. L.; King, B. T.; Foster, K. N.; Cairns, B. A.; Carter, J. E. Demonstration of the safety and effectiveness of the RECELL((R)) System combined with split-thickness meshed autografts for the reduction of donor skin to treat mixed-depth burn injuries. Burns 2019, 45 (4), 772-782. DOI: 10.1016/j.burns.2018.11.002.
(6) Manning, L.; Ferreira, I B.; Gittings, P.; Hiew, J.; Ryan, E.; Baba, M.; Raby, E.; Carville, K.; Norman, P. E.; Davis, W. A.; et al. Wound healing with “spray-on” autologous skin grafting (ReCell) compared with standard care in patients with large diabetes-related foot wounds: an open-label randomised controlled trial. Int Wound J 2021. DOI: 10.1111/iwj.13646.
(7) Spray-on skin technology saving lives of many in Australia and around the world. BioScience Managers, 2021. https://biosciencemanagers.com/spray-on-skin-technology-saving-lives-of-many-in-australia-and-around-the-world/ (accessed 2023 Aug. 1, 2023).
(8) Hoch, E.; Mcbirney, S.; Engel, C.; Piquado, T. Mitigating the Effects of Blast-Related Burn Injuries from Prolonged Field Care to Rehabilitation and Resilience. In Ninth Department of Defense International State-of-the-Science Meeting (SoSM) on Blast Injury Research, Arlington, V A, 2020; RAND Corporation.
(9) Lairet, J. R.; Bebarta, V. S.; Burns, C. J.; Lairet, K. F.; Rasmussen, T. E.; Renz, E. M.; King, B. T.; Fernandez, W.; Gerhardt, R.; Butler, F.; et al. Prehospital interventions performed in a combat zone: A prospective multicenter study of 1,003 combat wounded. J Trauma Acute Care 2012, 73, S38-S42. DOI: 10.1097/TA.0b013e3182606022.
(10) Murray, C. K. Epidemiology of infections associated with combat-related injuries in Iraq and Afghanistan. J Trauma 2008, 64 (3 Suppl), S232-238. DOI: 10.1097/TA.0b013e318163c3f5 From NLM Medline.
(11) Vig, K.; Chaudhari, A.; Tripathi, S.; Dixit, S.; Sahu, R.; Pillai, S.; Dennis, V. A.; Singh, S. R. Advances in Skin Regeneration Using Tissue Engineering. Int J Mol Sci 2017, 18 (4). DOI: 10.3390/ijms18040789.
(12) Chua, A. W.; Khoo, Y. C.; Tan, B. K.; Tan, K. C.; Foo, C. L.; Chong, S. J. Skin tissue engineering advances in severe burns: review and therapeutic applications. Burns Trauma 2016, 4, 3. DOI: 10.1186/s41038-016-0027-y.
(13) Barrientos, S.; Stojadinovic, O.; Golinko, M. S.; Brem, H.; Tomic-Canic, M. Growth factors and cytokines in wound healing. Wound Repair and Regeneration 2008, 16 (5), 585-601. DOI: 10.1111/j.1524-475X.2008.00410.x.
(14) Widjaja, W.; Tan, J.; Maitz, P. K. M. Efficacy of dermal substitute on deep dermal to full thickness burn injury: a systematic review. ANZ J Surg 2017, 87 (6), 446-452. DOI: 10.1111/ans.13920.
(15) Yildirimer, L.; Thanh, N. T.; Seifalian, A. M. Skin regeneration scaffolds: a multimodal bottom-up approach. Trends Biotechnol 2012, 30 (12), 638-648. DOI: 10.1016/j.tibtech.2012.08.004.
(16) Driver, V. R.; Lavery, L. A.; Reyzelman, A. M.; Dutra, T. G.; Dove, C. R.; Kotsis, S. V.; Kim, H. M.; Chung, K. C. A clinical trial of Integra Template for diabetic foot ulcer treatment. Wound repair and regeneration: official publication of the Wound Healing Society [and] the European Tissue Repair Society 2015, 23 (6), 891-900. DOI: 10.1111/wrr.12357.
(17) Marston, W. A.; Hanft, J.; Norwood, P.; Pollak, R.; Dermagraft Diabetic Foot Ulcer Study, G. The efficacy and safety of Dermagraft in improving the healing of chronic diabetic foot ulcers: results of a prospective randomized trial. Diabetes Care 2003, 26 (6), 1701-1705. DOI: 10.2337/diacare.26.6.1701.
(18) Stupp, S. I.; Clemons, T. D.; Carrow, J. K.; Sai, H.; Palmer, L. C. Supramolecular and Hybrid Bonding Polymers. Isr J Chem 2020, 60 (1-2), 124-131. DOI: 10.1002/ijch.202000005.
(19) Webber, M. J.; Tongers, J.; Newcomb, C. J.; Marquardt, K. T.; Bauersachs, J.; Losordo, D. W.; Stupp, S. I. Supramolecular nanostructures that mimic VEGF as a strategy for ischemic tissue repair. P Natl Acad Sci USA 2011,108 (33), 13438-13443. DOI: 10.1073/pnas.1016546108.
(20) Sather, N. A.; Sai, H.; Sasselli, I. R.; Sato, K.; Ji, W.; Synatschke, C. V.; Zambrotta, R. T.; Edelbrock, J. F.; Kohlmeyer, R. R.; Hardin, J. O.; et al. 3D Printing of Supramolecular Polymer Hydrogels with Hierarchical Structure. Small 2021, 17 (5). DOI: ARTN 2005743 10.1002/smll.202005743.
(21) Bury, M. I.; Fuller, N. J.; Clemons, T. D.; Sturm, R. M.; Morrison, C. D.; Lisy-Snow, D. C.; Nolan, B. G.; Tarczynski, C.; Ayello, E. M. T.; Boyce, A.; et al. Self-Assembling Nanofibers Inhibit Inflammation in a Murine Model of Crohn's-Disease-Like Ileitis. Advanced Therapeutics 2021, 4 (4), 2000274.
(22) Edelbrock, A. N.; Clemons, T. D.; Chin, S. M.; Roan, J. J. W.; Bruckner, E. P.; Alvarez, Z.; Edelbrock, J.; Wek, K. S.; Stupp, S. I Superstructured Biomaterials Formed by Exchange Dynamics and Host-Guest Interactions in Supramolecular Polymers. Advanced Science 2021, 2004042.
(23) Lewis, J. A.; Freeman, R.; Carrow, J. K.; Clemons, T. D.; Palmer, L. C.; Stupp, S. I. Transforming Growth Facto beta-1 Binding by Peptide Amphiphile Hydrogels. Acs Biomater Sci Eng 2020, 6 (8), 4551-4560. DOI: 10.1021/acsbiomaterials.0c00679.
(24) Craik, D. J.; Fairlie, D. P.; Liras, S.; Price, D. The Future of Peptide-based Drugs. Chem Biol Drug Des 2013, 81 (1), 136-147. DOI: 10.1111/cbdd.12055.
(25) Tang, L.; Persky, A. M.; Hochhaus, G.; Meibohm, B. Pharmacokinetic aspects of biotechnology products. J Pharm Sci 2004, 93 (9), 2184-2204. DOI: 10.1002/jps.20125.
(26) Tanaka, S.; Hamada, Y.; Yokoyama, Y.; Yamamoto, H.; Kogo, M. Osteopontin-derived synthetic peptide SVVYGLR upregulates functional regeneration of oral and maxillofacial soft-tissue injury. Jpn Dent Sci Rev 2021, 57, 174-181. DOI: 10.1016/j.jdsr.2021.09.002.
(27) Uchinaka, A.; Kawaguchi, N.; Ban, T.; Hamada, Y.; Mori, S.; Maeno, Y.; Sawa, Y.; Nagata, K.; Yamamoto, H. Evaluation of dermal wound healing activity of synthetic peptide SVVYGLR. Biochem Biophys Res Commun 2017, 491 (3), 714-720. DOI: 10.1016/j.bbrc.2017.07.124 From NLM Medline.
(28) Hamed, S.; Bennett, C. L.; Demiot, C.; Ullmann, Y.; Teot, L.; Desmouliere, A. Erythropoietin, a novel repurposed drug: an innovative treatment for wound healing in patients with diabetes mellitus. Wound repair and regeneration: official publication of the Wound Healing Society [and] the European Tissue Repair Society 2014, 22 (1), 23-33. DOI: 10.1111/wrr.12135.
(29) Galeano, M.; Altavilla, D.; Cucinotta, D.; Russo, G. T.; Calo, M.; Bitto, A.; Marini, H.; Marini, R.; Adamo, E. B.; Seminara, P.; et al. Recombinant human erythropoietin stimulates angiogenesis and wound healing in the genetically diabetic mouse. Diabetes 2004, 53 (9), 2509-2517. DOI: 10.2337/diabetes.53.9.2509.
(30) Menacho-Melgar, R.; Decker, J. S.; Hennigan, J. N.; Lynch, M. D. A review of lipidation in the development of advanced protein and peptide therapeutics. J Control Release 2019, 295, 1-12. DOI: 10.1016/j.jconrel.2018.12.032.
(31) Wrighton, N. C.; Farrell, F. X.; Chang, R.; Kashyap, A. K.; Barbone, F. P.; Mulcahy, L. S.; Johnson, D. L.; Barrett, R. W.; Jolliffe, L. K.; Dower, W. J. Small peptides as potent mimetics of the protein hormone erythropoietin. Science 1996, 273 (5274), 458-463. DOI: DOI 10.1126/science.273.5274.458.
(32) Li, J.; Chen, J.; Kirsner, R. Pathophysiology of acute wound healing. Clin Dermatol 2007, 25 (1), 9-18. DOI: 10.1016/j.clindermatol.2006.09.007.
(33) Eming, S. A.; Martin, P.; Tomic-Canic, M. Wound repair and regeneration: Mechanisms, signaling, and translation. Sci Transl Med 2014, 6 (265). DOI: ARTN 265sr6 10.1126/scitranslmed.3009337.
(34) Xiao, Y.; Reis, L. A.; Feric, N.; Knee, E. J.; Gu, J. H.; Cao, S. W.; Laschinger, C.; Londono, C.; Antolovich, J.; McGuigan, A. P.; et al. Diabetic wound regeneration using peptide-modified hydrogels to target re-epithelialization. P Natl Acad Sci USA 2016, 113 (40), E5792-E5801. DOI: 10.1073/pnas.1612277113.
(35) Lee, S.; Kim, M. S.; Jung, S. J.; Kim, D.; Park, H. J.; Cho, D. ERK activating peptide, AES16-2M promotes wound healing through accelerating migration of keratinocytes. Sci Rep-Uk 2018, 8. DOI: ARTN 14398 10.1038/s41598-018-32851-y.
(36) Kim, M. S.; Song, J.; Park, S.; Kim, T. S.; Park, H. J.; Cho, D. The Wound Healing Peptide, AES16-2M, Ameliorates Atopic Dermatitis In Vivo. Molecules 2021, 26 (4). DOI: 10.3390/molecules26041168.
(37) Preslar, A. T.; Parigi, G.; McClendon, M. T.; Sefick, S. S.; Moyer, T. J.; Haney, C. R.; Waters, E. A.; MacRenaris, K. W.; Luchinat, C.; Stupp, S. I.; et al. Gd(III)-labeled peptide nanofibers for reporting on biomaterial localization in vivo. Acs Nano 2014, 8 (7), 7325-7332. DOI: 10.1021/nn502393u.
(38) Sleep, E.; Cosgrove, B. D.; McClendon, M. T.; Preslar, A. T.; Chen, C. H.; Sangji, M. H.; Perez, C. M. R.; Haynes, R. D.; Meade, T. J.; Blau, H. M.; et al. Injectable biomimetic liquid crystalline scaffolds enhance muscle stem cell transplantation. Proc Natl Acad Sci USA 2017, 114 (38), E7919-E7928. DOI: 10.1073/pnas.1708142114.

What is claimed is:

1. A composition, comprising:
a plurality of non-covalently bonded peptide amphiphiles, wherein each of the peptide amphiphiles comprises:
a hydrophobic alkyl tail moiety,
a hydrophobic peptide portion comprising one or more hydrophobic amino acid residues, and
a hydrophilic portion comprising one or more hydrophilic amino acid residues,
wherein about 0 mol % to about 100 mol % of the plurality of peptide amphiphiles are functionalized with one or more bioactive components, and said one or more bioactive components are selected from the group consisting of bioactive epitope peptide sequence EMP1 (GGTYSCHFGPLTWVCKPQGG (SEQ ID NO.: 1)), bioactive epitope peptide ERK receptor activating REGRT (SEQ ID NO.2), bioactive epitope peptide sequence SVVYGLR (SEQ ID NO.: 3), bioactive epitope peptide sequence, QHREDGS (SEQ ID NO.4), cell adhesion peptide sequence, RGDS (SEQ ID NO: 5), bioactive wound healing peptide sequence, GLLSGINAEWPC (SEQ ID NO: 6), antioxidant moiety lipoic acid, antioxidant moiety protocatechuic acid (PCA), and antioxidant moiety glutathione tripeptide.

2. The composition of claim 1, wherein the peptide amphiphiles are in an aqueous solution.

3. The composition of claim 1, further comprising a biological material selected from the group consisting of biological fluids, biological cells, and biological tissues.

4. The composition of claim 1, further comprising a biological material selected from the group consisting of an autologous material, an allogeneic material, a xenogeneic material and a biomimetic synthetic of a biological material.

5. The composition of claim 3, wherein the biological material comprises autologous cells.

6. The composition of claim 1, having a shear modulus of from about 2 Pa to about 20,000 Pa.

7. The composition of claim 1, wherein the hydrophobic alkyl tail moiety has from about 6 to 22 carbon atoms carbon atoms.

8. The composition of claim 1, wherein the hydrophobic portion comprises one or more hydrophobic amino acid residues of amino acids selected from the group consisting of glycine, alanine, valine, leucine, phenylalanine, cysteine, isoleucine, and combinations thereof.

9. The composition of claim 1, wherein the hydrophilic portion comprises one or more hydrophilic amino acid residues of amino acids selected from the group consisting of serine, asparagine, glutamine, threonine, arginine, lysine, histidine, aspartic acid, and glutamic acid and combinations thereof.

10. The composition of claim 1, wherein the hydrophilic portion further comprises a component selected from the group consisting of a beta sheet forming region, amino acid residues of non-natural amino acids and uncharged hydrophilic moieties.

11. The composition of claim 1, wherein the hydrophobic alkyl tail moiety is a hexadecyl group, the one or more hydrophobic amino acid residues comprise alanine and valine, and the one or more hydrophilic amino acid residues comprise glutamic acid.

12. The composition of claim 1, wherein one or more of said peptide amphiphiles are selected from the group consisting of:

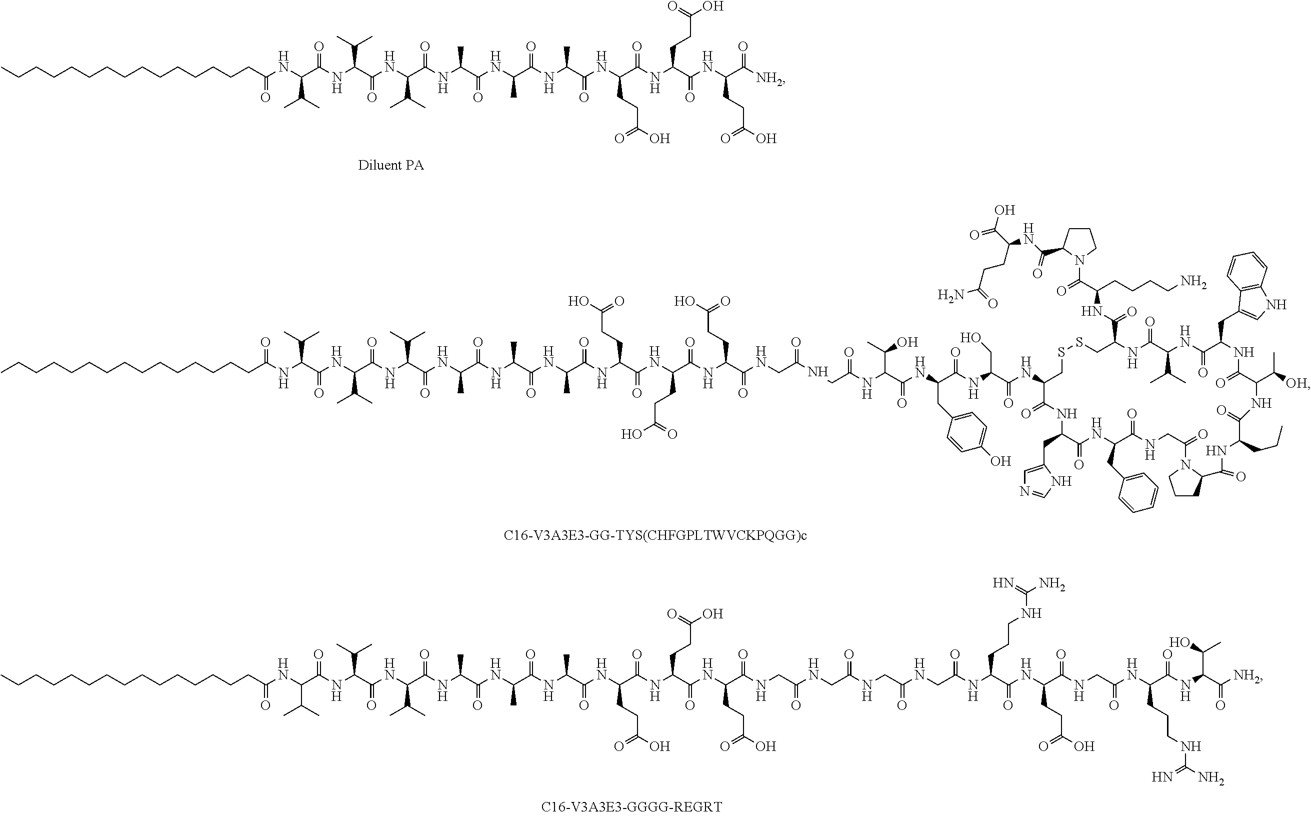

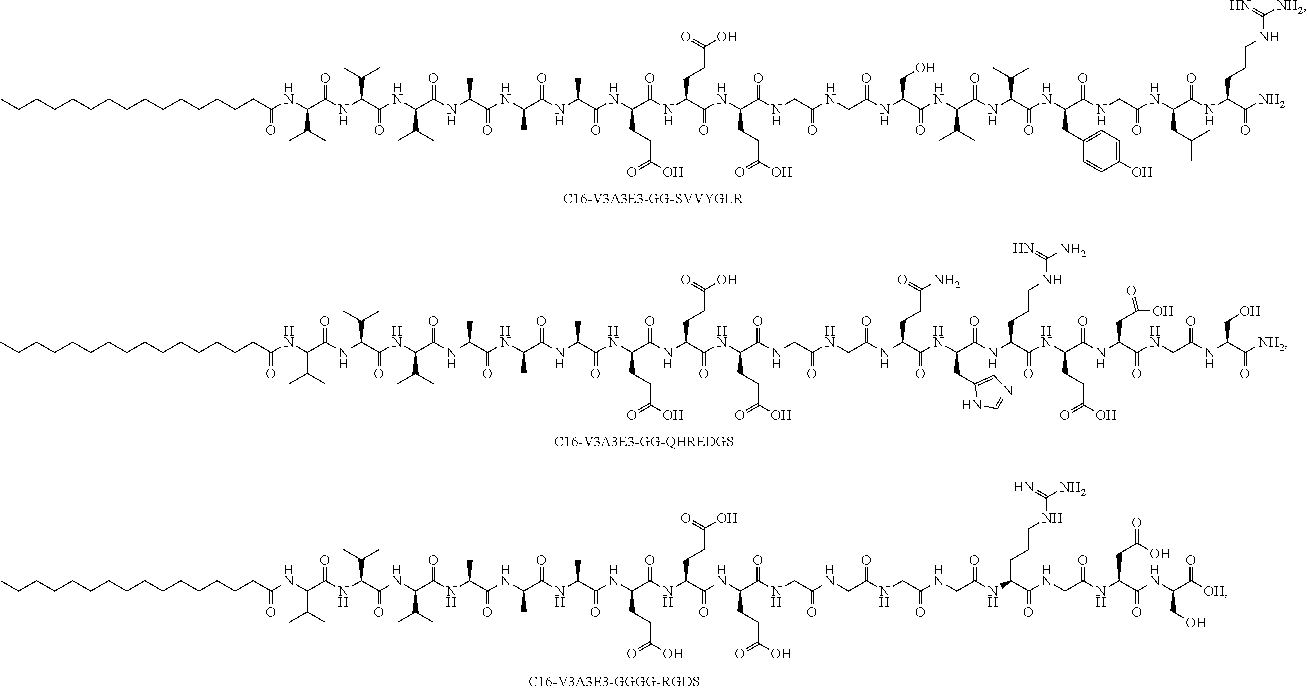

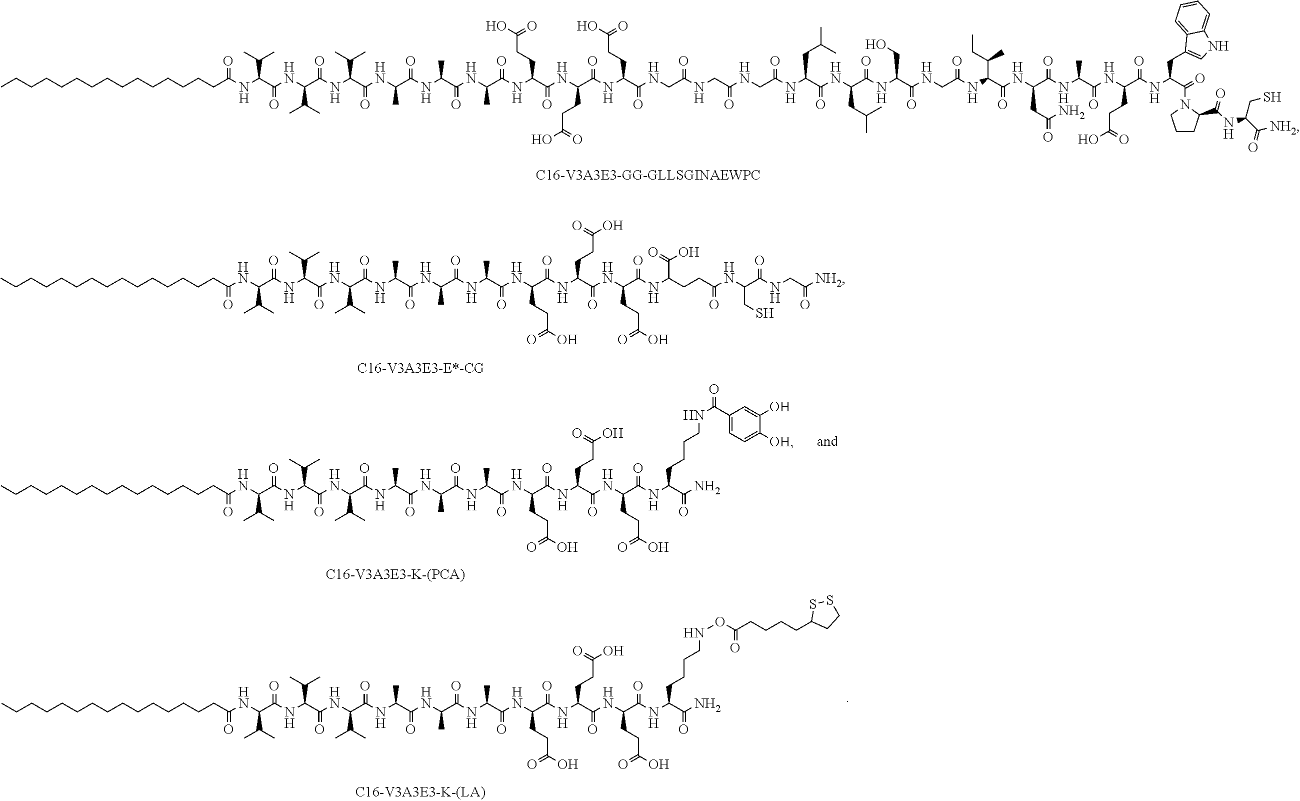

13. The composition of claim 1, wherein about 5 mol % to about 100 mol % of the peptide amphiphiles are functionalized with the one or more bioactive components.

14. The composition of claim 13, wherein the composition is capable of forming a bioactive scaffold.

15. The composition of claim 1, wherein the composition comprises an aqueous buffer.

16. The composition of claim 1, wherein the composition is capable of forming a hydrogel in the presence of water.

17. The composition of claim 1, wherein the composition is a thixotropic fluid.

18. A supramolecular polymer nanofiber scaffold comprising a plurality of the non-covalently bonded peptide amphiphiles as claimed in claim 1.

19. The supramolecular polymer nanofiber scaffold of claim 18, wherein at least some of the peptide amphiphiles are selected from the group consisting of 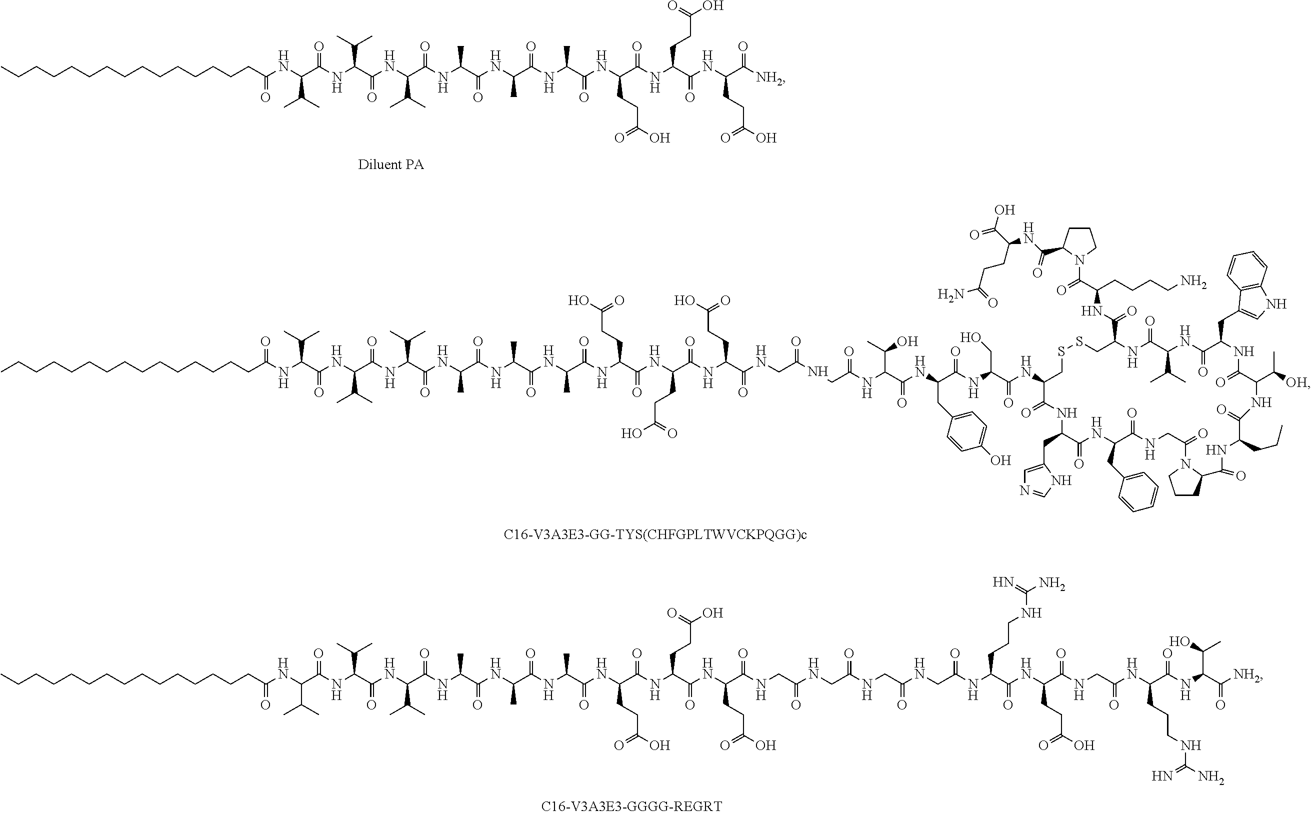 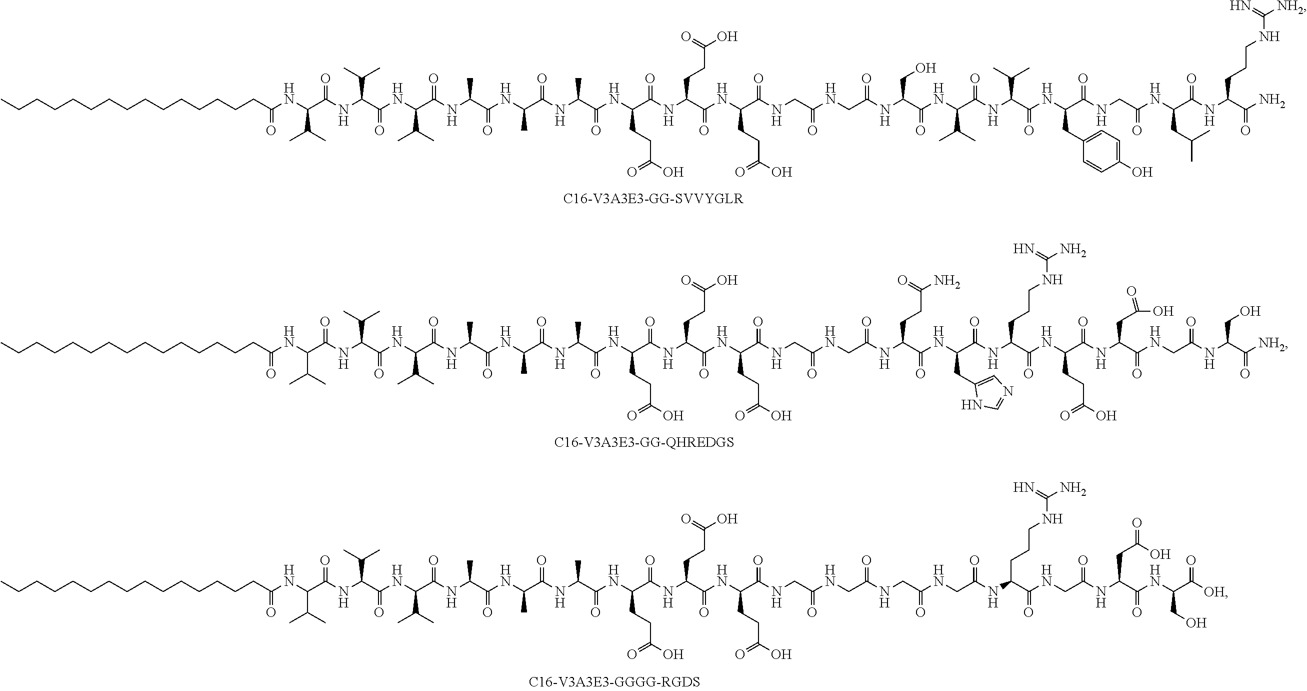 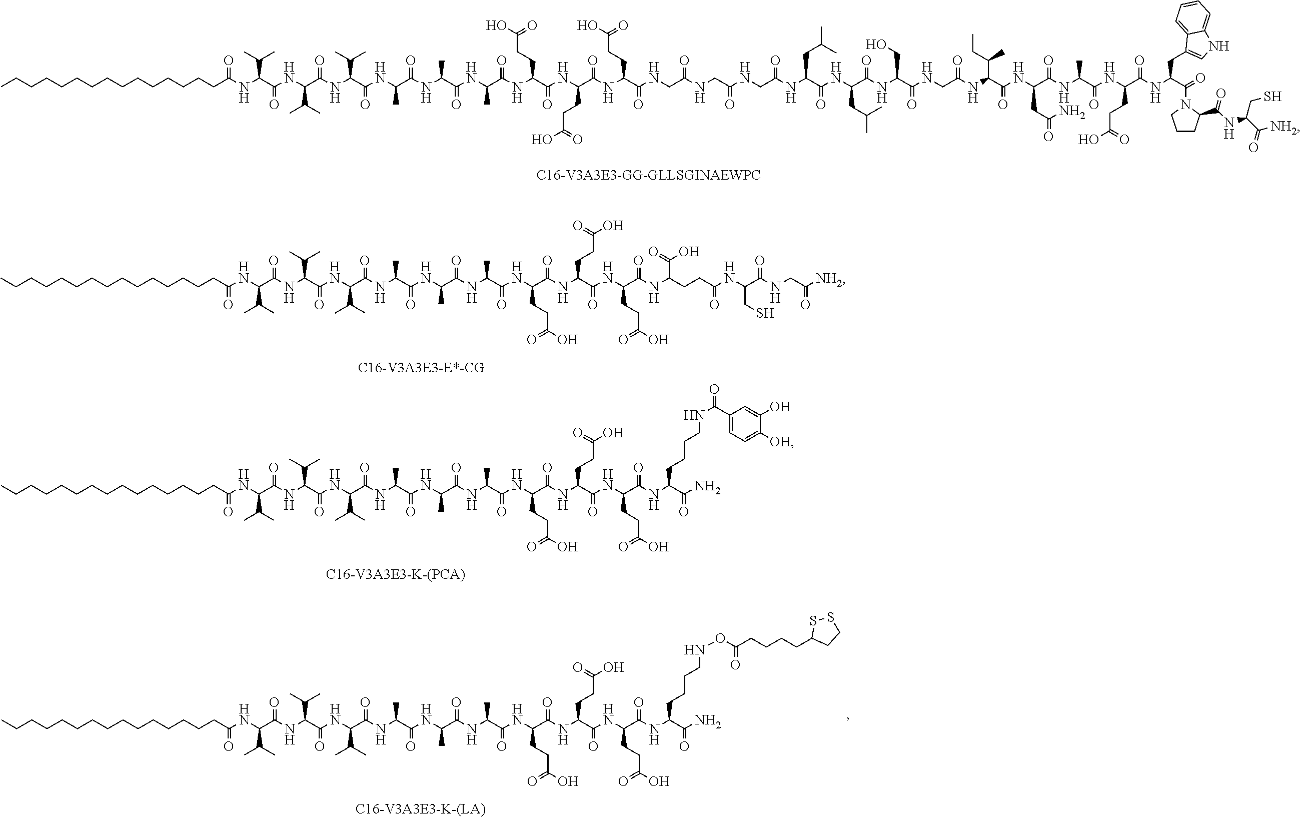
and any combination thereof.

20. The supramolecular polymer nanofiber scaffold of claim 18, wherein the supramolecular polymer nanofiber scaffold is configured to reversibly disassemble or break in response to high shear conditions and to self-assemble when returned to lower shear conditions.

21. A kit comprising:
the composition of claim 1 and a delivery device selected from the group consisting of a syringe, a dropper, a film, a 3D bioprinter, and a sprayer.

22. A method for promoting skin cell tissue regeneration comprising steps of:
preparing a composition of claim 1,
optionally, collecting a biological material,
mixing an aqueous composition, and the optional biological material with the composition of claim 1 to produce an aqueous suspension or solution, and
administering the suspension or a solution to a patient using high shear conditions.
